SEQUENCE LISTING (32 sequences)

SEQ ID NO: 1 - DNA, 21 bp, Artificial Sequence
  Primer Sequence
actcctacgggaggcagcagt

SEQ ID NO: 2 - DNA, 18 bp, Artificial Sequence
  Primer Sequence
attaccgcggctgctggc

SEQ ID NO: 3 - DNA, 18 bp, Artificial Sequence
  Primer Sequence
ctcctggaaacgggtggt

SEQ ID NO: 4 - DNA, 18 bp, Artificial Sequence
  Primer Sequence
gctgcctcccgtaggagt

SEQ ID NO: 5 - DNA, 19 bp, Artificial Sequence
  Primer Sequence
ggttctgagaggaggtccc

SEQ ID NO: 6 - DNA, 18 bp, Artificial Sequence
  Primer Sequence
gctgcctcccgtaggagt

SEQ ID NO: 7 - DNA, 19 bp, Artificial Sequence
  Primer Sequence
agcagtagggaatcttcca

SEQ ID NO: 8 - DNA, 17 bp, Artificial Sequence
  Primer Sequence
caccgctacacatggag

SEQ ID NO: 9 - DNA, 30 bp, Artificial Sequence
  Primer Sequence
tttgggctacacacgtgctacaatggacaa

SEQ ID NO: 10 - DNA, 24 bp, Artificial Sequence
  Primer Sequence
aacaactttatgggatttgcwtga

SEQ ID NO: 11 - DNA, 21 bp, Artificial Sequence
  Primer Sequence
attcatagtctagttggaggt

SEQ ID NO: 12 - DNA, 20 bp, Artificial Sequence
  Primer Sequence
cctgaactgagagaatttga

SEQ ID NO: 13 - DNA, 20 bp, Artificial Sequence
  Primer Sequence
atcagagggggataacactt

SEQ ID NO: 14 - DNA, 21 bp, Artificial Sequence
  Primer Sequence
actctcatccttgttcttctc

SEQ ID NO: 15 - DNA, 23 bp, Artificial Sequence
  Primer Sequence
agcttagaagcagctattcattc

SEQ ID NO: 16 - DNA, 21 bp, Artificial Sequence
  Primer Sequence
ggatacacctttcggtctctc

SEQ ID NO: 17 - DNA, 18 bp, Artificial Sequence
  Primer Sequence
actcctacgggaggcagc

SEQ ID NO: 18 - DNA, 24 bp, Artificial Sequence
  Primer Sequence
gcttcttaagtcaggtaccgtcat

SEQ ID NO: 19 - DNA, 25 bp, Artificial Sequence
  Primer Sequence
gggagcaaacaggattagataccct

SEQ ID NO: 20 - DNA, 27 bp, Artificial Sequence
  Primer Sequence
tgcaccatctgtcactctgttaacctc

SEQ ID NO: 21 - DNA, 19 bp, Artificial Sequence
  Primer Sequence
gtgccagcmgccgcggtaa

SEQ ID NO: 22 - DNA, 23 bp, Artificial Sequence
  Primer Sequence
gcctcaagggcacaacctccaag

SEQ ID NO: 23 - DNA, 22 bp, Artificial Sequence
  Primer Sequence
tcgcatcgatgaagaacgcagc

SEQ ID NO: 24 - DNA, 25 bp, Artificial Sequence
  Primer Sequence
tcttttcctccgcttattgatatgc

SEQ ID NO: 25 - DNA, 19 bp, Artificial Sequence
  Primer Sequence
attgctggccttttcattg

SEQ ID NO: 26 - DNA, 22 bp, Artificial Sequence
  Primer Sequence
cgcctagacgctctcttcttat

SEQ ID NO: 27 - DNA, 25 bp, Artificial Sequence
  Primer Sequence
ctgttagtgcgggagttcaaattct

SEQ ID NO: 28 - DNA, 22 bp, Artificial Sequence
  Primer Sequence
aacacctgacctttcgcgtgta

SEQ ID NO: 29 - DNA, 22 bp, Artificial Sequence
  Primer Sequence
caccaggttgattctgcctgac

SEQ ID NO: 30 - DNA, 21 bp, Artificial Sequence
  Primer Sequence
cctctccggaaccaaaccctg

SEQ ID NO: 31 - DNA, 20 bp, Artificial Sequence
  Primer Sequence
ccgggtatctaatccggttc

SEQ ID NO: 32 - DNA, 20 bp, Artificial Sequence
  Primer Sequence
ctcccagggtagaggtgaaa

(19) United States
(12) Patent Application Publication
(10) Pub. No.: US 2013/0052172 A1
(43) Pub. Date: Feb. 28, 2013

(54) METHODS FOR DIAGNOSING AND TREATING CARDIAC DEFECTS

(71) Applicant: John Edward Baker, Wauwatosa, WI (US)

(72) Inventor: John Edward Baker, Wauwatosa, WI (US)

(21) Appl. No.: 13/441,854

(22) Filed: Apr. 7, 2012

Related U.S. Application Data
(60) Provisional application No. 61/527,738, filed on Aug. 26, 2011.

(51) Int. Cl. A61K 38/14; C12Q 1/68; C40B 30/10; C12N 1/20; C07K 9/00; A61K 31/7036; C07H 15/232; C07H 15/238; C07K 7/62; C07K 7/58; A61K 35/74; A61P 31/00; A61P 9/00; A61K 38/12; C40B 30/04

(52) U.S. Cl. 424/93.45; 506/9; 435/6.12; 506/12; 435/252.1; 514/2.3; 530/322; 514/37; 514/39; 536/13.2; 536/14; 530/319; 530/320; 424/93.4

(57) ABSTRACT

The present disclosure defines a method for identifying and/or treating risk and/or occurrence of cardiac defect. As is shown herein, microbiomes are reproducibly and detectably associated with cardiac defect risk factors and changes to the microbiome can directly alter cardiac defect risk. The present disclosure demonstrates that microbial signatures can be used to characterize components of microbiomes that associate with altered risk or occurrence of cardiac defects and to identify treatments to reduce risk or severity of cardiac defects.

### RELATED APPLICATIONS

This application claims priority to U.S. Provisional Patent Application Ser. No. 61/527,738, filed Aug. 26, 2011; the entirety of which is hereby incorporated by reference.

### GOVERNMENT SUPPORT

The United States Government has provided grant support utilized in the development of the present invention. In particular, National Institutes of Health grant numbers AI080363 and HL54075 have supported development of this invention. The United States Government may have certain rights in the invention.

### BACKGROUND

Coronary heart disease is a major health issue in the United States and worldwide, and is a major contributor to heart attacks, also known as Myocardial Infarction (MI), a leading cause of death worldwide. Underlying causes are often quite advanced when coronary heart disease is detected. It is commonly understood that the severity of coronary heart disease can be managed through use of medication and lifestyle modifications. Moreover, cardiovascular diseases, disorders, and conditions in general present some of the most significant challenges in the health care industry.

### SUMMARY

The present invention encompasses the recognition that reproducible and detectable changes in microbiome composition and/or activity are associated with incidence and/or risk of cardiac defect. The present invention permits identification and/or characterization of microbial signatures reflecting such changes, and also provides systems for using such microbial signatures, for example to assess and/or treat incidence and/or risk of cardiac defect.

In some embodiments, a microbial signature comprises a level or levels of one or more microbes or components or products thereof and is sufficient to distinguish or characterize a microbiome of an individual with an incidence and/or risk of cardiac defect to be characterized and/or identified relative to a microbiome of an individual with no incidence and/or risk of cardiac defect, or with a known incidence and/or risk of cardiac defect. For example, in some embodiments, microbial signatures obtained from gastrointestinal microbiomes of individuals at increased risk for cardiac defect are sufficient to diagnose individuals as having increased risk when compared with microbial signatures of gastrointestinal microbiomes of individuals who are not at increased risk for cardiac defect.

In accordance with the present invention, microbial signatures are defined for particular microbiota samples relative to appropriate reference microbiota samples. In some embodiments, particular microbiota samples share a common feature of incidence and/or risk of cardiac defect that is not shared by reference microbiota samples. In some embodiments, particular microbiota samples differ from reference microbiota samples in that they are samples of a different source. In some embodiments particular microbiota samples differ from reference microbiota samples in that microbiota reference samples are historical microbiota samples of a same or a different source.

In certain embodiments, the present disclosure provides methods for identifying and/or characterizing incidence and/or risk of cardiac defect comprising providing a reference microbial signature that correlates with extent and/or degree of cardiac defect and determining a microbial signature present in a microbiota sample from an individual whose incidence and/or risk of cardiac defect is to be identified or characterized. In some embodiments, a microbiota sample comprises a sample of one or more types of microbes found in a gastrointestinal tract of a subject. In some embodiments, the microbial signature comprises a level or set of levels of one or more 16S rRNA gene sequences of one or more types of microbes. In some embodiments, the microbial signature comprises a level or set of levels of one or more metabolites of one or more types of microbes.

In certain embodiments, the present disclosure provides methods for monitoring a patient scheduled to receive or having received a cardiac procedure comprising providing a reference microbial signature that correlates with extent and/or degree of cardiac defect and determining a microbial signature present in a microbiota sample from a patient whose incidence and/or risk of cardiac defect is to be identified or characterized. In some embodiments, the microbiota sample comprises a sample of one or more types of microbes found in a gastrointestinal tract of a subject. In some embodiments, the microbial signature comprises a level or set of levels of one or more 16S rRNA gene sequences of one or more types of microbes. In some embodiments, the microbial signature comprises a level or set of levels of one or more metabolites of one or more types of microbes.

In certain embodiments, the present disclosure provides methods for identifying and/or characterizing microbial signatures correlated with incidence and/or risk of cardiac defect comprising determining a first set of levels of one or more types of microbes or components or products thereof in a first collection of microbiota samples, where each sample in the first collection of microbiota samples shares a common feature of incidence and/or risk of cardiac defect, determining a second set of levels of the one or more types of microbes or components or products thereof in a second collection of microbiota samples, which second collection of microbiota samples does not share the common feature of incidence and/or risk of cardiac defect but is otherwise comparable to the first set of microbiota samples, and identifying a microbial signature comprising levels within the first or second set that correlates with presence or absence of the common feature of incidence and/or risk of cardiac defect. In some embodiments, microbiota samples are obtained from host organisms and the common feature of incidence and/or risk of cardiac defect comprises incidence of coronary heart disease in host organisms. In some embodiments, microbiota samples are obtained from host organisms and the common feature of incidence and/or risk of cardiac defect comprises prior history of myocardial infarction in host organisms. In some embodiments, a common feature of incidence and/or risk of cardiac defect comprises exposure to a microbiome-altering agent having a known correlation with cardiac defect risk. In some embodiments, a level or set of levels of one or more types of microbes or components or products thereof comprises a level or set of levels of one or more microbial metabolites present in a microbiota sample.

In certain embodiments, the present disclosure provides methods for treating or reducing risk for cardiac defect in an individual by altering the microbiome of the individual, the methods comprising steps of administering to an individual suffering from or susceptible to a cardiac defect a microbiome-altering agent, such that the individual's microbiome is altered in a manner that correlates with altered severity of or risk for the cardiac defect. In some embodiments, the microbiome altering agent comprises one or more antibiotics. In some embodiments, the microbiome altering agent comprises one or more types of microbes.

In certain embodiments, the present disclosure provides compositions comprising microbiome altering agents that, when administered to an individual, alter the individual's microbiome in a manner correlated with reduced severity or risk of cardiac defect. In some embodiments, the compositions further comprise a pharmaceutically acceptable carrier. In certain embodiments, the compositions are provided in or as a food product, functional food or nutraceutical. In some embodiments, the compositions are in a unit dosage form, containing a unit dose amount for administration in accordance with a dosing regimen correlated with achievement of the reduced severity or risk of cardiac defect. In certain embodiments, the microbiome altering agent is or comprises bacterial cells. In some embodiments, the microbiome altering agent comprises or further comprises an antibiotic.

### BRIEF DESCRIPTION OF THE DRAWING

FIG. 1 shows a flow diagram illustrating the systems biology approach used as it relates to the interplay between the microbiota resident within the intestines, and microbial metabolites exported into the circulation of the host in the setting of myocardial infarction.

FIG. 2 presents a chart depicting primer sets specific for the 16S and 18S rRNA of particular microbial phylum, class, genus, and species (Methanobrevibacter smithii and L. plantarum) along with quantitative PCR reaction temperature.

FIG. 3 shows a bar graph illustrating microbial populations in the feces of vancomycin-treated rats. Log₁₀ microbial number per gram feces is graphed as a function of microbial type. Vancomycin administered orally (60 mg/kg/d) by addition to the drinking water altered abundance of microbial species present in feces and reduced total microbial numbers. The x-axis labels represent 3 microbial taxa, bacteria, fungi, and archaea. L. plantarum is part of the Bacilli class of bacteria. ND indicates not detected. Data are means±sd; n=6/group. * indicates P<0.01 vs. d0.

FIGS. 4A-4C show bar graphs illustrating the effect of vancomycin administration on myocardial infarction in rats. Infarct size is graphed as a function of antibiotic treatment. 4A) Vancomycin added to the drinking water (60 mg/kg/d) reduced infarct size (IS) in vivo. 4B) Vancomycin added directly to the coronary circulation of isolated hearts did not reduce IS in vitro. 4C) Vancomycin added to the drinking water (60 mg/kg/d) and then excluded from the coronary circulation of isolated hearts reduced IS. Data are means±sd; n=6/group. LV indicates left ventricle. Reduction in IS was similar for in vitro and in vivo studies (A, C). * indicates P<0.01 vs. control.

FIG. 5 presents a bar graph illustrating that vancomycin treatment confers cardioprotection in rats within 48 h, and the effect is lost by 72 h after cessation of treatment. Infarct size is graphed as a function of vancomycin treatment over time. Vancomycin was added to the drinking water (60 mg/kg/d) before heart excision for ischemia/reperfusion studies. Food and (vancomycin) water were fed ad libitum to all rats. Data are means±sd; n=4. * indicates P<0.05 vs. control.

FIGS. 6A-6B present bar graphs illustrating that intestinal microbiota mediate cardioprotection via leptin in rats. 6A) Quantified changes in 11 of 23 cytokines Levels of cytokines are graphed in pg/ml as a function of vancomycin treatment. 6B) Leptin reconstitution reversed cardioprotection by vancomycin. Rats were treated with leptin (0.12 g/kg i.v.) at 24 and 12 h before myocardial ischemia/reperfusion. Infarct size (IS) and recovery left ventricular developed pressure (LVDP) are graphed as a function of leptin and vancomycin treatment. Data are means±sd; n=6/group. * indicates P<0.05 vs. control.

FIGS. 7A-7C show graphs illustrating that probiotic juice decreased leptin and protected against myocardial infarction in rats. Dahl S rats were treated with probiotic juice (15 ml/rat/d) for 14 d before blood leptin analysis and myocardial ischemia/reperfusion. FIG. 7A shows a bar graph in which IS and recovery left ventricular developed pressure (LVDP) are graphed as a function of leptin and probiotic juice treatment. Probiotic juice reduced myocardial infarction that was reversed by leptin reconstitution (0.12 g/kg at 24 and 12 h before ischemia). FIG. 7B shows a bar graph in which blood leptin levels in pg/ml are graphed as a function of leptin and probiotic juice treatment. Leptin levels in blood were decreased following probiotic juice treatment. FIG. 7C shows a scatter plot in which Log₁₀ L. plantarum per gram rat feces is plotted as a function of leptin and probiotic juice treatment. L. plantarum levels increased in feces of probiotic juice-treated rats using quantitative PCR of 16S rRNA. Limit of detection for L. plantarum is 3 log₁₀/g feces. Data are means±sd; n=6/group. * indicates P<0.01 vs. control.

FIGS. 8A-8B present bar graphs of IS (8A) or blood leptin levels in pg/ml (8B) as a function of leptin and probiotic juice treatment. Probiotic juice treatment protected against myocardial infarction and decreased leptin levels in rats. Dahl S rats were treated with either probiotic juice (15 ml/rat/d, ˜1.5×10⁹ L. plantarum/rat/d), irradiated (35 kGy) probiotic juice, or vehicle (water, 92.8 mg/ml glucose, 42.2 μg/ml NaCl, 464 μg/ml KCl, and 4 mg albumin) for 14 d or injected with 0.12 μg/kg leptin at 24 and 12 h, or both, before heart excision for ischemia/reperfusion studies. 8A) IS of the hearts of treated rats. 8B) Blood plasma of rats in 8A was collected immediately before ischemia/reperfusion and analyzed for leptin levels. Data are means±sd; n=6/group. * indicates P<0.02, ⁺ indicates P<0.01 vs. control.

FIGS. 9A-9E illustrate receptors, intracellular survival pathways and ATP-dependent potassium channels activated by microbial metabolites in rats. Bar graphs plot IS or recovery LVDP as a function of treatment with vancomycin and intracellular signaling inhibitors. Hearts isolated from control- and vancomycin-treated rats were perfused with pharmacological inhibitors of (9A) JAK-2, (9B) Akt, (9C) p42/44 MAPK, (9D) p38 MAPK and (9E) K_{ATP} channels prior to ischemia/reperfusion. Results are expressed as infarct size and recovery of mechanical function post reperfusion. Data are mean±sd; n=6/group. * indicates P<0.05 vs. control.

FIG. 10 shows a bar graph illustrating reduction of infarct size in rats by vancomycin and thrombopoietin is additive. Infarct size (IS) is plotted as a function of vancomycin and thrombopoietin treatment. Rats were untreated, or treated with vancomycin alone (15 mg/kg/day for 72 hours prior to ischemia), thrombopoietin (Tpo) alone (0.025 pg/kg i.v. for 15 minutes prior to ischemia) or vancomycin plus thrombopoietin prior to myocardial ischemia/reperfusion. Data are mean±sd; n=9/group. * indicates P<0.05 vs. control.

FIG. 11 shows a bar graph illustrating rat strain differences in microbial populations in feces of vancomycin treated rats. Microbial abundance is plotted as a function of vancomycin treatment, microbial type, and rat strain. Vancomycin administered orally altered abundance of microbial type and reduced total microbial number. This effect is rat strain dependent. WAG indicates WAG/RijCmcr rats. SD indicates Sprague Dawley rats. DSS indicates Dahl S rats. Data are mean±sd; n=9/group. * indicates P<0.05 vs. Day 0.

FIGS. 12A-12C present bar graphs illustrating rat strain differences in myocardial infarction with antibiotic treatment. Infarct size (IS) is plotted as a function of antibiotic treatment for WAG/RijCmcr (12A), Sprague Dawley (12B) and Dahl S (12C) rats. Antibiotics were added to the drinking water. Rats were untreated, treated with Vancomycin (60 mg/kg/day), or treated with a combination of Streptomycin (120 mg/kg/day), Neomycin (60 mg/kg/day), Bacitracin (120 mg/kg/day), and Polymyxin B (60 mg/kg/day). Data are mean±sd; n=6/group. * indicates P<0.05 vs. control. LV indicates left ventricle.

FIGS. 13A-13B demonstrate the impact of antibiotics on infarct size and bacterial abundance. Antibiotics were added to the drinking water of Dahl S rats in the following concentrations: 120 mg/kg/day streptomycin, 60 mg/kg/day polymyxin B, 120 mg/kg/day bacitracin, 60 mg/kg/day neomycin, or 60 mg/kg/day vancomycin. FIG. 13A shows a bar graph in which Infarct size (IS) is plotted as a function of antibiotic treatment. FIG. 13B shows a graphical table illustrating antibiotic induced changes in bacterial taxa and correlating those changes to cardiac protection.

FIG. 14 presents a bar graph illustrating microbial populations in the feces of antibiotic treated rats. Microbes per gram feces are plotted as a function of microbial taxon. A mixture of streptomycin, neomycin, polymyxin B, and bacitracin administered orally altered abundance of microbial species present in feces and reduce total microbial numbers. The X-axis labels show taxons of microbes grouped by bacteria, fungi, and archaea. ND indicates not detected. Data are mean±sd; n=6/group. * indicates P<0.05 vs. day 0.

FIGS. 15A-15C show bar graphs illustrating the effect of administration of an antibiotic mixture on myocardial infarction. Infarct size (IS) is plotted as a function of antibiotic treatment. 15A) Antibiotics added to the drinking water reduced infarct size in vivo. 15B) Antibiotic added directly to the coronary circulation of isolated hearts did not reduce infarct size in vitro. 15C) Antibiotic added to the drinking water and then excluded from the coronary perfusate reduced infarct size. Data are means±sd; n=6/group. * indicates P<0.01, vs. control. AAR indicates area at risk. LV indicates left ventricle.

FIGS. 16-18 present box and whisker plots illustrating the effect of antibiotics on intestinal microbiota metabolites of tryptophan (16), phenylalanine (17) and tyrosine (18). Amount of each metabolite is plotted as a function of antibiotic treatment. Mean values are represented by plus signs. Median values are represented by horizontal bars. Top and bottom boxes represents the upper and lower quartiles. Whiskers represent the maximum and minimum values. Open circle represents an extreme data point. Data are means±sd, n=8/group, * indicates P<0.05 vs. day 0, NS indicates not significant.

FIG. 19 presents a bar graph illustrating the effect of intestinal microbial metabolites on infarct size in rats. Infarct size is plotted as a function of treatment with intestinal microbial metabolites. Reduction of infarct size with vancomycin was abolished by pretreatment with metabolites of all three or each individual amino acid: phenylalanine, tryptophan and tyrosine. Rats treated with vancomycin were administered metabolites of phenylalanine (F), (Trans-cinnamate+phenylacetate+3-phenylpropionate), tryptophan (W), (Indole-3-acetate+3-indoxyl sulfate+L-kynurenine+3-indolepropionate), or tyrosine (Y), (4-hydroxyphenylpyruvate+p-hydroxyphenyllactate) intravenously or orally prior to ischemia/reperfusion studies. Data are mean±sd, n=6/group, * indicates P<0.05 vs. control. iv indicates intravenous. o indicates oral.

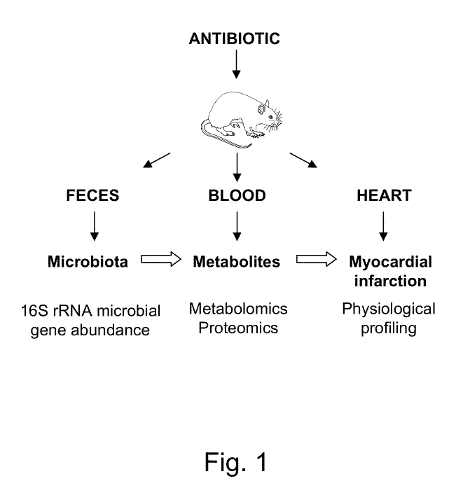

### DEFINITIONS

Antibiotic: As used herein, the term “antibiotic agent” means any of a group of chemical substances, isolated from natural sources or derived from antibiotic agents isolated from natural sources, having a capacity to inhibit growth of, or to destroy bacteria, and other microorganisms, used chiefly in treatment of infectious diseases. Examples of antibiotic agents include, but are not limited to; Amikacin; Amoxicillin; Ampicillin; Azithromycin; Azlocillin; Aztreonam; Aztreonam; Carbenicillin; Cefaclor; Cefepime; Cefetamet; Cefinetazole; Cefixime; Cefonicid; Cefoperazone; Cefotaxime; Cefotetan; Cefoxitin; Cefpodoxime; Cefprozil; Cefsulodin; Ceftazidime; Ceftizoxime; Ceftriaxone; Cefuroxime; Cephalexin; Cephalothin; Cethromycin; Chloramphenicol; Cinoxacin; Ciprofloxacin; Clarithromycin; Clindamycin; Cloxacillin; Co-amoxiclavuanate; Dalbavancin; Daptomycin; Dicloxacillin; Doxycycline; Enoxacin; Erythromycin estolate; Erythromycin ethyl succinate; Erythromycin glucoheptonate; Erythromycin lactobionate; Erythromycin stearate; Erythromycin; Fidaxomicin; Fleroxacin; Gentamicin; Imipenem; Kanamycin; Lomefloxacin; Loracarbef; Methicillin; Metronidazole; Mezlocillin; Minocycline; Mupirocin; Nafcillin; Nalidixic acid; Netilmicin; Nitrofurantoin; Norfloxacin; Ofloxacin; Oxacillin; Penicillin G; Piperacillin; Retapamulin; Rifaxamin, Rifampin; Roxithromycin; Streptomycin; Sulfamethoxazole; Teicoplanin; Tetracycline; Ticarcillin; Tigecycline; Tobramycin; Trimethoprim; Vancomycin; combinations of Piperacillin and Tazobactam; and their various salts, acids, bases, and other derivatives. Anti-bacterial antibiotic agents include, but are not limited to, aminoglycosides, carbacephems, carbapenems, cephalosporins, cephamycins, fluoroquinolones, glycopeptides, lincosamides, macrolides, monobactams, penicillins, quinolones, sulfonamides, and tetracyclines.

Antibacterial agents also include antibacterial peptides. Examples include but are not limited to abaecin; andropin; apidaecins; bombinin; brevinins; buforin II; CAP18; cecropins; ceratotoxin; defensins; dermaseptin; dermcidin; drosomycin; esculentins; indolicidin; LL37; magainin; maximum H5; melittin; moricin; prophenin; protegrin; and or tachyplesins.

Cardiac Defect: As is described herein, “cardiac defect” is any disease, disorder, condition, or event involving hearts and/or blood vessels. In some embodiments, a cardiac defect comprises an increased risk for any disease, disorder, condition, or event involving hearts and blood vessels. In some embodiments, a cardiac defect comprises any departure from normal operation of hearts and blood vessels. In some embodiments, an individual having a cardiac defect is suffering from or susceptible to any disease, disorder, condition, or event involving hearts and blood vessels. In some embodiments, a cardiac defect is or comprises a heart attack or myocardial infarction, or injury therefrom. In some embodiments, a cardiac defect is or comprises a stroke, or injury therefrom. In some embodiments, a cardiac defect is or comprises an ischemic event, or injury therefrom. In some embodiments, a cardiac defect is or comprises coronary artery disease, or injury therefrom. In some embodiments, a cardiac defect is or comprises atherosclerosis, or injury therefrom.

Carrier: As used herein, the terms “carrier” refers to a pharmaceutically acceptable (e.g., safe and non-toxic for administration to a human) carrier or diluting substance useful for the preparation of a pharmaceutical formulation. Exemplary diluents include sterile water, bacteriostatic water for injection (BWFI), a pH buffered solution (e.g. phosphate-buffered saline), sterile saline solution, Ringer's solution or dextrose solution.

Combination Therapy: The term “combination therapy”, as used herein, refers to those situations in which two or more different pharmaceutical agents are administered in overlapping regimens so that the subject is simultaneously exposed to both agents.

Comparable: Sufficiently similar to permit comparison, but differing in at least one feature.

Correlates: The term “correlates”, as used herein, has its ordinary meaning of “showing a correlation with”. Those of ordinary skill in the art will appreciate that two features, items or values show a correlation with one another if they show a tendency to appear and/or to vary, together. In some embodiments, a correlation is statistically significant when its p-value is less than 0.05; in some embodiments, a correlation is statistically significant when its p-value is less than 0.01. In some embodiments, correlation is assessed by regression analysis. In some embodiments, a correlation is a correlation coefficient.

Differentiates: The term “differentiates”, as used herein, indicates defining or distinguishing from other entities (e.g., comparable entities). In some embodiments, differentiates means distinguishing from other types with which present together in source and/or sample.

Dosing regimen: A “dosing regimen” (or “therapeutic regimen”), as that term is used herein, is a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which are separated from one another by a time period of the same length; in some embodiments, a dosing regime comprises a plurality of doses and at least two different time periods separating individual doses. In some embodiments, the therapeutic agent is administered continuously over a predetermined period. In some embodiments, the therapeutic agent is administered once a day (QD) or twice a day (BID).

Incidence: As will be understood from context, an “incidence” of a disease, disorder, or condition and/or an undesirable cardiac event (together, “incidence” of a cardiac defect) comprises an individual suffering from and/or previously having suffered from a disease, disorder, or condition, or event (cardiac defect).

Infarct: The term “infarct” is typically used in the art to refer to a tissue lesion and/or area of tissue death resulting from a local lack of oxygen due to blood supply obstruction.

Ischemia: The term “ischemia” is typically used in the art to refer to restriction of blood flow to tissues. Ischemia prevents tissues from receiving necessary oxygen and nutrients carried in blood. In some embodiments, ischemia is a reduction of blood supply in arteries. In some embodiments, ischemia is a reduction of blood supply in a coronary artery. In some embodiments, ischemia is a reduction of blood supply in blood vessels. In some embodiments, reduction of blood supply is a reduction of blood supply of 0.1, 1, 2, 3, 4, 5, 6, 7, 8, 9, 10% or more of a non-reduced blood supply. In some embodiments, a reduction of blood supply is a total lack of blood supply.

Metabolite: The term “metabolite” as used herein, refers to any compound formed by in vivo biotransformation of any chemical by any metabolic process. In some embodiments, metabolites are produced by oxidation. In some embodiments, metabolites are produced by reduction. In some embodiments, metabolites are produced by hydrolysis. In some embodiments, metabolites are produced by or conjugation. In some embodiments, metabolites comprise polypeptides. In some embodiments, metabolites comprise carbohydrates. In some embodiments, metabolites comprise small molecules. In some embodiments, metabolites are produced by cells of a multicellular organism. In some embodiments, metabolites are produced by single-celled organisms. In some embodiments, metabolites are produced by microbial cells.

Microbe: The term “microbe” is typically used in the art to refer to a microscopically small organisms such as a bacterium, fungus, protozoan, or virus. In some embodiments, a microbe is a bacterium, archaeon, unicellular fungus (e.g., yeast), alga, or a protozoa (e.g., plasmodia as a malaria pathogen). In some embodiments, microbes are characterized according to their kingdom. In some embodiments, microbes are characterized according to their phylum. In some embodiments, microbes are characterized according to their class. In some embodiments, microbes are characterized according to their family. In some embodiments, microbes are characterized according to their genus. In some embodiments, microbes are characterized according to their species. In some embodiments, microbes are characterized according to their subspecies. In some embodiments, microbes are characterized according to their strain. Occasionally additional taxonomic class(es), e.g., serovars or serotypes, are used for differentiating microbes, such as bacteria, included within a subspecies. Serovars and serotypes are distinguished by their different types of attachment behavior at a cell membrane. In some embodiments, genus and species are utilized to identify and/or characterize a microbe (e.g., in a sample). In some embodiments, subspecies, serotype and/or strain are utilized to identify and/or characterize a microbe (e.g., in a sample). Alternatively or additionally, in some embodiments, a microbe (e.g., in a sample) is identified and/or characterized using one or more distinguishing characteristics such as pathogenicity (i.e., an ability to bring on a particular illness), or resistance to one or more antibiotics, metabolic profiles, morphology, etc.

Microbial Types: As will be understood from the context, the term “microbial types” or “types of microbes” is used herein to indicate a grouping of microbes with a common feature. In some embodiments, a microbial type is a group of microbes sharing a common detectable feature. In some embodiments, a common detectable feature is or comprises presence or amount of a particular DNA sequence. In some embodiments, a common detectable feature is or comprises presence or amount of a particular RNA transcript. In some embodiments, a common detectable feature is or comprises presence or amount of a polypeptide (e.g., a microbially-produced polypeptide). In some embodiments, a common detectable feature is or comprises presence or amount of a metabolite (e.g., a microbially-produced metabolite). In some embodiments, a common detectable feature is or comprises presence or level of an enzymatic activity (e.g., of a microbial enzyme). In some embodiments, microbes of a common type are microbes of a particular classification, according to standard taxonomy. Those of skill in the art will understand that the term “microbial type” as used herein is not restricted to a specific degree of resolution; different features may be detected using technologies that achieve different levels of resolution. In some embodiments, microbes of a common type are microbes of the same microbial kingdom. In some embodiments, microbes of a common type are microbes of the same microbial phylum. In some embodiments, microbes of a common type are microbes of the same microbial class. In some embodiments, microbes of a common type are microbes of the same microbial family. In some embodiments, microbes of a common type are microbes of the same microbial genus. In some embodiments, microbes of a common type are microbes of the same microbial species. In some embodiments, microbes of a common type are microbes of the same microbial subspecies. In some embodiments, microbes of a common type are microbes of the same microbial serovar. In some embodiments microbes of a common type are microbes of the same microbial serotype. In some embodiments, microbes of a common type are microbes of the same strain.

Microbiome Altering Agent: As used herein, the term “microbiome altering agent” refers to an agent that alters the microbiome in an individual (e.g., by altering absolute or relative level and/or activity of one or more microbes present in the microbiome). In some embodiments, microbiome altering agents comprise an agent that increases relative levels(s) of one or more types of microbes in the microbiome. In some embodiments, microbiome altering agents comprise an agent that decreases relative level(s) of one or more types of microbes in the microbiome. In some embodiments, microbiome altering agents comprise an agent that increases absolute level(s) of one or more types of microbes in the microbiome, including by adding one or more types of microbes. In some embodiments, microbiome altering agents comprise an agent that decreases absolute level(s) of one or more types of microbes in the microbiome, including by substantially removing (e.g., by killing) one or more types of microbes. In some embodiments, microbiome altering agents comprise an agent that increases total number of microbes in the microbiome. In some embodiments, microbiome altering agents comprise an agent that decreases total number of microbes in the microbiome. In some embodiments, microbiome altering agents comprise chemicals. In some embodiments, microbiome altering agents comprise antimicrobials. In some embodiments, microbiome altering agents comprise antibiotics. In some embodiments, microbiome altering agents comprise non-absorbable antibiotics. In some embodiments, microbiome altering agents comprise bacitracin, neomycin, polymyxin B, streptomycin, and/or vancomycin, or combinations thereof. In some embodiments, microbiome altering agents comprise microbes. In some such embodiments, microbiome altering agents comprise bacteria. In some embodiments, microbiome altering agents comprise probiotic bacteria. In some embodiments, microbiome altering agents comprise Lactobacillus plantarum. In some embodiments, microbiome altering agents comprise Bifidobacterium lactis. In some embodiments, microbiome altering agents comprise antimicrobial peptides. In some embodiments, microbiome altering agents comprise anti-fungals. In some embodiments, microbiome altering agents comprise bacteriophages.

Polypeptide: The term “polypeptide” as used herein refers a sequential chain of amino acids linked together via peptide bonds. The term is used to refer to an amino acid chain of any length, but one of ordinary skill in the art will understand that the term is not limited to lengthy chains and can refer to a minimal chain comprising two amino acids linked together via a peptide bond. As is known to those skilled in the art, polypeptides may be processed and/or modified.

Probiotic: As is described herein, “probiotic” is any microbial type that is associated with health benefits in a host organism and/or reduction of risk and/or symptoms of a disease, disorder, condition, or event in a host organism. In some embodiments, probiotics are formulated in a food product, functional food or nutraceutical. In some embodiments, probiotics are types of bacteria. Examples of bacterial probiotics include Bacillus coagulans, Bifidobacterium animalis, Bifidobacterium animalis DN 173 010, Bifidobacterium animalis subsp. lactis Bb-12, Bifidobacterium breve Yakult, Bifidobacterium infantis, Bifidobacterium infantis 35624, Bifidobacterium lactis, Bifidobacterium lactis HN019 (DR10), Bifidobacterium longum BB536, Enterococcus LAB SF 68, Escherichia coli Nissle 1917, Lactobacillus acidophilus, Lactobacillus acidophilus LA-5, Lactobacillus acidophilus NCFM, Lactobacillus casei DN-114 001, Lactobacillus casei CRL431, Lactobacillus casei F19, Lactobacillus casei Shirota, Lactobacillus GG, Lactobacillus johnsonii, Lactobacillus johnsonii La1 (Lj1), Lactobacillus lactis, Lactococcus lactis L1A, Lactobacillus paracasei, Lactobacillus plantarum, Lactobacillus plantarum 299V, Lactobacillus reuteri, Lactobacillus reuteri ATTC 55730, Lactobacillus rhamnosus Lactobacillus rhamnosus ATCC 53013 (LGG), Lactobacillus rhamnosus LB21 and/or Lactobacillus salivarius UCC118. In some embodiments, probiotics are types of fungi. Examples of fungal probiotics include Saccharomyces cerevisiae (boulardii) lyo.

Protein: The term “protein” as used herein refers to one or more polypeptides that function as a discrete unit. If a single polypeptide is the discrete functioning unit and does not require permanent or temporary physical association with other polypeptides in order to form the discrete functioning unit, the terms “polypeptide” and “protein” may be used interchangeably. If the discrete functional unit is comprised of more than one polypeptide that physically associate with one another, the term “protein” refers to the multiple polypeptides that are physically coupled and function together as the discrete unit.

Reference: As will be understood from context, a reference sample or individual is one that is sufficiently similar to a particular sample or individual of interest to permit a relevant comparison. In some embodiments, information about a reference sample is obtained simultaneously with information about a particular sample. In some embodiments, information about a reference sample is historical. In some embodiments, information about a reference sample is stored for example in a computer-readable medium. In some embodiments, comparison of a particular sample of interest with a reference sample establishes identity with, similarity to, or difference of the particular sample of interest relative to the reference.

Risk: As will be understood from context, a “risk” of a disease, disorder condition, or event (cardiac defect) comprises a likelihood that a particular individual will develop a disease, disorder, or condition, and/or will suffer an undesirable cardiac event (together, that the person will suffer a cardiac defect). In some embodiments, risk is expressed as a percentage. In some embodiments, risk is from 0, 1, 2, 3, 4, 5, 6, 7, 8, 9, 10 up to 100%. In some embodiments risk is expressed as a risk relative to a risk associated with a reference sample or group of reference samples. In some embodiments, a reference sample or group of reference samples have a known risk of a disease, disorder, condition and/or event (cardiac defect). In some embodiments a reference sample or group of reference samples are from individuals comparable to a particular individual. In some embodiments, relative risk is 0, 1, 2, 3, 4, 5, 6, 7, 8, 9, 10 or more.

Sample: As used herein, the term “sample” refers to a biological sample obtained or derived from a source of interest, as described herein. In some embodiments, a source of interest comprises an organism, such as an animal or human. In some embodiments, a biological sample comprises biological tissue or fluid. In some embodiments, a biological sample may be or comprise bone marrow; blood; blood cells; ascites; tissue or fine needle biopsy samples; cell-containing body fluids; free floating nucleic acids; sputum; saliva; urine; cerebrospinal fluid, peritoneal fluid; pleural fluid; feces; lymph; gynecological fluids; skin swabs; vaginal swabs; oral swabs; nasal swabs; washings or lavages such as a ductal lavages or broncheoalveolar lavages; aspirates; scrapings; bone marrow specimens; tissue biopsy specimens; surgical specimens; feces, other body fluids, secretions, and/or excretions; and/or cells therefrom, etc. In some embodiments, a biological sample is or comprises cells obtained from an individual. In some embodiments, obtained cells are or include cells from an individual from whom the sample is obtained. In some embodiments, obtained cells are or include microbial cells of an individual's microbiome. In some embodiments, a sample is a “primary sample” obtained directly from a source of interest by any appropriate means. For example, in some embodiments, a primary biological sample is obtained by methods selected from the group consisting of biopsy (e.g., fine needle aspiration or tissue biopsy), surgery, collection of body fluid (e.g., blood, lymph, feces etc.), etc. In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example nucleic acids or proteins extracted from a sample or obtained by subjecting a primary sample to techniques such as amplification or reverse transcription of mRNA, isolation and/or purification of certain components, etc.

Substantially: As used herein, the term “substantially” refers to a qualitative condition of exhibiting total or near-total extent or degree of a characteristic or property of interest. Those of ordinary skill in the biological arts will appreciate that biological and chemical phenomena rarely, if ever, go to completion and/or proceed to completeness or achieve or avoid an absolute result. The term “substantially” is therefore used herein to capture a potential lack of completeness inherent in many biological and chemical phenomena.

Susceptible to: An individual who is “susceptible to” a disease, disorder, or condition and/or an undesirable cardiac event (together, that the individual is “susceptible to” a cardiac defect) is not presently suffering from and/or may not exhibit symptoms of the disease, disorder, condition, or event. In some embodiments, an individual who is susceptible to a disease, disorder, condition, or event (for example, cardiac defect) may be characterized by one or more of the following: (1) a genetic mutation associated with development of the disease, disorder, condition, and/or event; (2) a genetic polymorphism associated with development of the disease, disorder, condition, and/or event; (3) increased and/or decreased expression and/or activity of a protein associated with the disease, disorder, condition, and/or event; (4) habits and/or lifestyles associated with development of the disease, disorder, condition, and/or event; (5) a family history of the disease, disorder, condition, and/or event; (6) reaction to certain microbes; (7) exposure to certain chemicals. In some embodiments, an individual who is susceptible to a disease, disorder, condition, and/or event, or event will develop the disease, disorder, condition, and/or event. In some embodiments, an individual who is susceptible to a disease, disorder, condition, and/or event will not develop the disease, disorder, condition, and/or event.

Suffering from: An individual who is “suffering from” a disease, disorder, or condition and/or is “suffering from” an undesirable cardiac event (together, that the individual is “suffering from” a cardiac defect) has presently been diagnosed with and/or presently exhibits one or more symptoms of the disease, disorder, condition, or event.

Therapeutically effective amount: As used herein, the term “therapeutically effective amount” refers to an amount of a microbiome altering agent which confers a therapeutic effect on a treated subject, at a reasonable benefit/risk ratio applicable to any medical treatment. A therapeutic effect may be objective (i.e., measurable by some test or marker) or subjective (i.e., subject gives an indication of or feels an effect). In particular, a “therapeutically effective amount” refers to an amount of a therapeutic agent effective to treat, ameliorate, or prevent a desired disease or condition, or to exhibit a detectable therapeutic or preventative effect, such as by ameliorating symptoms associated with a disease, preventing or delaying onset of a disease, and/or also lessening severity or frequency of symptoms of a disease. A therapeutically effective amount is commonly administered in a dosing regimen that may comprise multiple unit doses. For any particular therapeutic agent, a therapeutically effective amount (and/or an appropriate unit dose within an effective dosing regimen) may vary, for example, depending on route of administration, on combination with other agents. Also, a specific therapeutically effective amount (and/or unit dose) for any particular patient may depend upon a variety of factors including what disorder is being treated; disorder severity; activity of specific agents employed; specific composition employed; age, body weight, general health, sex and diet of a patient; time of administration, route of administration; treatment duration; and like factors as is well known in the medical arts.

Transcript: As used herein, the term “transcript” refers to a molecule as transcribed or alternately as processed in one or more steps of splicing, ect.

Unit dose: The term “unit dose”, as used herein, refers to a discrete administration of a pharmaceutical agent, typically in the context of a dosing regimen.

### DETAILED DESCRIPTION OF CERTAIN EMBODIMENTS

### Cardiac Defect

Cardiac defects are a major cause of illness and death worldwide. As is described herein, cardiac defects can arise from a disease, disorder, condition, and/or undesirable event involving hearts and/or blood vessels. In many embodiments, cardiac defects pertain to and/or stem from a change in heart and/or coronary artery physiology. In some embodiments, a cardiac defect arises from and/or is associated with a cardiac disease or event, such as angina, atherosclerosis, cardiac arrhythmias, cardiomyopathy, congestive heart failure, coronary heart disease, endocarditis, hypertensive heart disease, ischaemic heart disease, ischemia, ischemia/reperfusion injury, left ventricular hypertrophy, myocardial infarction, myocarditis, reperfusion injury, stroke, and/or sudden cardiac death. In some embodiments, cardiac disease is naturally occurring. In some embodiments, cardiac disease is artificially induced.

### Atherosclerosis and Coronary Heart Disease

Many forms of cardiac defects stem from or originate with atherosclerosis. Atherosclerosis is a condition in which artery walls thicken as a result of accumulation of fatty materials such as cholesterol. Deposition of fatty materials on artery walls induces a sustained immune response resulting in plaque formation and stenosis. This immune response is characterized by an attraction of platelets and monocytes to areas of cholesterol accumulation. Monocytes then differentiate into foam cells, which have a high content of internal lipid vesicles. As foam cells die, and the immune response is further induced, more immune cells are recruited resulting in areas where dead high-fatty content immune cells have accumulated, or plaques. Stenosis, or narrowing of arteries, can result from repeated plaque rupture and repair.

Coronary heart disease describes severe atherosclerosis in the heart and coronary artery. In coronary heart disease, plaque buildup caused by atherosclerosis results in reduced blood flow to areas of the heart.

Symptoms of coronary heart disease include, but are not limited to angina, shortness of breath, increased heartbeat, weakness or dizziness, sweating and/or nausea, and any combination thereof. It is commonly understood in the art that conditions in arteries associated with atherosclerosis and coronary heart disease predispose individuals to a variety of cardiac conditions including myocardial infarction.

Current methods for diagnosing coronary heart disease include physical exam, blood tests, ankle/brachial index, CT-scan, angiography, electrocardiography, stress testing, and/or echocardiography.

During a physical exam, a stethoscope can be used to detect abnormal heart sounds indicating poor blood flow due to plaque buildup. A weak or absent pulse can be a sign of a blocked artery.

Blood tests for coronary artery disease include but are not limited to tests for C-reactive protein, fibrinogen, homocysteine, cholesterol, lipoprotein (a), and/or natriuretic peptides or combinations thereof.

Ankle/brachial index compares blood pressure in a patient's ankle and arm to see how well their blood is flowing.

Through computer-generated pictures, CT scans can show hardening and narrowing of large arteries.

Angiography uses dye and X-rays to visualize plaque in arteries.

Electrocardiography records heart electrical activity. It shows how fast a patient's heart is beating and its rhythm (steady or irregular). An irregular or elevated heart beat can indicate narrowing of arteries.

Stress testing involves inducing stress on a patient's heart by, for example, having them walk or run on a treadmill while performing tests such as electrocardiography to test heart function under stress.

Echocardiography uses sound waves to create a moving picture that provides information about heart size and shape, in addition to indicating how well heart chambers and valves are working, and areas of poor blood flow.

Risk factors for atherosclerosis and coronary heart disease include but are not limited to hyperlipidemia, elevated levels of C-reactive protein, vitamin B₆ deficiency, diabetes mellitus, diet, inactivity, obesity, stress, hypertension, tobacco use, gender (e.g., male), age, family history, and/or drug use.

Treatments for atherosclerosis and coronary heart disease include but are not limited to lifestyle changes. Lifestyle changes include but are not limited increasing physical activity, smoking cessation, limiting alcohol consumption, maintaining a healthy weight, and consuming a diet low in saturated fats. Treatments for atherosclerosis and coronary heart disease also include but are not limited to treatment with medication including angiotensin II receptor blockers, angiotensin-converting enzyme (ACE) inhibitors, antiarrhythmics, antiplatelet drugs, aspirin, beta blockers, calcium channel blockers, digoxin, diuretics, statins, thrombolytics, and/or vasodilators, including nitroglycerin, or combinations thereof. In severe cases of coronary heart disease, treatments also include but are not limited to surgical interventions. Surgical interventions include but are not limited to angioplasty, insertion of stents, coronary artery bypass, and/or heart transplant, or combinations thereof.

In patients with hyperlipidemia, atherosclerosis and coronary heart disease can further be treated by treating a patient for hyperlipidemia. Treatments for hyperlipidemia include lifestyle changes, as described in the present disclosure, and medications including, but not limited to statins.

In patients with diabetes mellitus, atherosclerosis and coronary heart disease can further be treated by treating a patient for diabetes mellitus. Treatments for diabetes mellitus include lifestyle changes, as described in the present disclosure, and medications including, but not limited to alpha glucosidase inhibitors, biguanides, dipeptidyl peptidase inhibitors, or ergot alkaloids, insulin, meglitinides, sulfonylureas, and/or thiazolidinediones, or combinations thereof.

In patients with hypertension, atherosclerosis and coronary heart disease can further be treated by treating a patient for hypertension. Treatments for hypertension include lifestyle changes, as described in the present disclosure, and medications including, but not limited to alpha blockers, alpha-beta blockers, angiotensin II receptor blockers, angiotensin-converting enzyme (ACE) inhibitors, beta blockers, calcium channel blockers, central-acting agents, renin inhibitors, thiazide diuretics, and/or vasodilators or combinations thereof.

### Myocardial Infarction

Myocardial infarction (MI) is a leading cause of death in the United States and in most industrialized nations worldwide. MI occurs when cardiac blood flow is reduced and/or blocked, resulting in myocardial cell damage and/or death (myocardial infarct). As is commonly understood in the art, myocardial infarction is often precipitated by atherosclerosis and/or coronary heart disease, yet often is also the first noticed symptom of atherosclerosis and/or coronary heart disease. When plaques from atherosclerosis and/or coronary heart disease rupture, they can form blood clots that can block blood flow. When cardiac blood flow is returned, reperfusion injury can occur. Studies in animal models suggest that reperfusion injury accounts for up to 50% of final size of a myocardial infarct.

Anatomically, MI presents as one of two types: transmural and nontransmural. Transmural MI is characterized by ischemic necrosis of the full thickness of affected cardiac muscle and/or segments, extending from the endocardium through the myocardium to the epicardium. Nontransmural MI is defined as an area of ischemic necrosis that does not extend through the full thickness of myocardial wall segment and/or segments. In nontransmural MI, the area of ischemic necrosis is limited to the endocardium or to the endocardium and myocardium.

MI is also classified as one of six types according to its clinical features. Type 1 is spontaneous MI related to ischemia from a primary coronary event (e.g., plaque rupture, thrombotic occlusion). Type 2 is secondary to ischemia from a supply-and-demand mismatch. Type 3 is MI resulting in sudden cardiac death. Type 4a is MI associated with percutaneous coronary intervention. Type 4b is associated with in-stent thrombosis. Type 5 is MI associated with coronary artery bypass surgery.

Symptoms of myocardial infarction include, but are not limited to chest pressure heaviness and/or pain, left and/or right arm pain, lower jaw pain, neck pain, back pain, epigastrium pain, Levine's sign, a heartburn like feeling, sweating, nausea, vomiting, dizziness, light-headedness, weakness, fatigue, sleep disturbances, anxiety, shortness of breath, heart palpitations. Approximately one quarter of myocardial infarctions present without symptoms.

Current methods for diagnosing myocardial infarction include but are not limited to electrocardiography, blood tests and/or echocardiography.

Abnormalities in electrical activity usually occur with MI and electrocardiography can identify areas of heart muscle that are deprived of oxygen and/or areas of muscle that have died. One advantage of electrocardiography is that it is a rapid means of diagnosis. However, diagnosis from electrocardiography can be difficult when patients present with atypical symptoms or have abnormal electrical patterns.

Blood tests for diagnosing myocardial infarction assay for presence of cardiac enzymes. During MI, cardiac enzymes are released into the blood stream by dying heart muscles. Enzymes assayed include but are not limited to creatine kinase, troponin I, troponin T, and/or myoglobin or combinations thereof. These enzymes are typically elevated for several hours after MI.

Echocardiography can detect damaged areas of heart muscle. However, echocardiography cannot distinguish between recent and historical events and abnormalities may also be indicative of conditions other than MI.

Risk factors for MI are similar to risk factors for atherosclerosis and coronary heart disease. Risk factors include but are not limited to atherosclerosis, coronary heart disease, hyperlipidemia, diabetes mellitus, diet, inactivity, obesity, stress, hypertension, tobacco use, gender (e.g., male), advanced age, family history, and/or drug use.

Current methods for reducing risk of MI include treating causes of and/or risk factors for MI. In some embodiments, treating causes of and/or risk factors of MI comprises lifestyle changes as is described in the present disclosure. In some embodiments, treating causes of and/or risk factors of MI comprises treating atherosclerosis, and/or coronary heart disease, as is described in the present disclosure. In some embodiments, treating causes of and/or risk factors of MI comprises treating hyperlipidemia, as is described in the present disclosure. In some embodiments, treating causes of and/or risk factors of MI comprises treating diabetes mellitus, as is described in the present disclosure. In some embodiments, treating causes of and/or risk factors of MI comprises treating hypertension, as is described in the present disclosure.

Current methods for treating MI include administration of antiplatelet agents to prevent accumulation of platelets at clot sites, oxygen therapy to increase oxygen delivery to damaged tissues, and/or administration of nitrates. Nitrates serve as a vasodialator.

Long term effects from MI depend on severity of MI and size of damaged heart tissue. Long term effects can include, but are not limited to increased risk of aneurysms, increased risk of pericarditis, angina, increased risk for congestive heart failure, oedema, depression, loss of sex drive and/or erectile dysfunction, increased risk for a subsequent MI event, and/or an enlarged heart or combinations thereof.

### Animal Models of Myocardial Infarction

One way that MI is studied in animal models is by artificially producing ischemia/reperfusion as it occurs during myocardial infarction and then measuring infarct produced and recovery of mechanical function as left ventricular developed pressure (LVDP) relative to preischemic LVDP as measures of MI severity. Techniques for performing ischemia/reperfusion are well known in the art in vivo as described, for example, in “K(ATP) opener-induced delayed cardioprotection: involvement of sarcolemmal and mitochondrial K(ATP) channels, free radicals and MEK½” (Gross, E. et al., J. Mol. Cell. Cardiol. 35, 985-992, 2003). Techniques for performing ischemia/reperfusion are well known in the art in vitro as described, for example, “Resistance to myocardial ischemia in five rat strains: is there a genetic component of cardioprotection?” (Baker, J. et al., Am. J. Physiol. Heart Circ. Physiol., 2000).

### Stroke

Two of the most common types of strokes are ischemic strokes and hemorrhagic strokes. In ischemic strokes, a lack of oxygen flow to the brain can result in apoptosis and necrosis of brain tissue leading to infarction. Similar to cardiovascular ischemia, brain ischemia can be caused by various factors such as blood clots, thrombosis, embolism, blockage by atherosclerotic plaques, or other obstructions in the vasculature. Hypercholesterolemia, hypertension, diabetes, and obesity, among other things, have been identified as risk factors for ischemic strokes. Ischemic strokes are a leading cause of death of human beings worldwide.

Hemorrhagic strokes, which account for between about 10 and 20 percent of all strokes, are typically caused by a ruptured blood vessel in the brain. The rupture causes bleeding into the brain, where the accumulating blood can damage surrounding neural tissues.

The stroke episode, regardless of its cause, results in neural cell death, especially at the location of the obstruction or hemorrhage. In addition, biochemical reactions that occur subsequent to the stroke episode in the vasculature may lead to edema, hemorrhagic transformation, and a further compromise in neurological tissue. The neurological damage and neuron cell death that result from a stroke can be physically and mentally debilitating to an individual. Among other things, a stroke can result in problems with emotional control, awareness, sensory perception, speech, hearing, vision, cognition, movement and mobility, and can cause paralysis.

### Microbiome

A human body typically contains ten times as many microbial (and particularly bacterial) cells as it has human cells. Many or most of such microbes are harmless, or even beneficial, to their human host. Increasingly, research demonstrates that such microbes play a significant role in maintaining and/or promoting human health. Gastrointestinal bacteria are a well studied example. These bacteria are thought to provide a variety of important functions including but not limited to aiding in carbohydrate digestion, regulating of intestinal cell growth, repressing pathogenic microbial growth, promoting development of intestinal mucosal immunity, metabolizing carcinogens, and preventing allergies and inflammatory bowel diseases.

All types and abundances of microbes in a particular environment comprise a microbiome. As microbes are nearly ubiquitous, microbiomes exist in most locations. In some embodiments a microbiome comprises microbes associated with any defined location. In some embodiments a microbiome comprises microbes associated with a living organism, or a particular portion, organ, tissue, or component thereof. In some embodiments, such an organism is a non-human multicellular organism. In some embodiments, such an organism is an animal. In some embodiments, an animal is a mouse, rat, cat, dog, rabbit, horse, cow, goat, sheep, frog, fish and/or pig. In some embodiments, an animal is a non-human primate. In some embodiments, an organism is a human.

Content (e.g., type and/or abundance of microbes present) and/or behavior (e.g., production of one or more markers, rate of respiration and/or proliferation, extent of migration, etc) of a microbiome can be shaped by local environments; in some embodiments; a single organism contains multiple different microbiomes, for example in different locations within or portions of their bodies. The human microbiome project (http://commonfund.nih.gov/hmp/) is characterizing the microbial communities found at several different sites on the human body, including nasal passages, oral cavities, skin, gastrointestinal tract, and urogenital tract. In some embodiments, a microbiome for use in accordance with the present invention is one associated with a particular site or location (e.g., tissue or organ) of an organism's body. In some embodiments a microbiome comprises microbes associated with skin. In some embodiments a microbiome comprises microbes associated with teeth. In some embodiments a microbiome comprises microbes associated with oral mucosa. In some embodiments a microbiome comprises microbes associated with nasal passages. In some embodiments a microbiome comprises microbes associated with a urogenital system. In some embodiments a microbiome comprises microbes associated with a gastrointestinal tract.

In some embodiments, a microbiome comprises a single microbe. In some embodiments a microbiome comprises between 1 and a trillion or more individual microbes. In some embodiments, a microbiome comprises a single type of microbe. In some embodiments, a microbiome comprises between 1 and a million or more types of microbes. In some embodiments, a microbiome comprises between 500 and 5,000 types of microbes. In some embodiments, a microbiome comprises between 1000 and 2,000 types of microbes. Types of microbes that reside in the intestines are generally described at the phylum, class, order and family levels. In some embodiments, there are between 1000-1500 types of bacteria in gastrointestinal tract microbiomes.

### Microbiome Changes

The present invention teaches that microbiome composition and/or activity, and more particularly that changes in microbiome composition and/or activity can be informative about particular environmental conditions, and specifically about the health status of a host organism. The invention presented herein encompasses the finding that microbiome composition and/or activity can change in detectable and reproducible ways that are correlated with risk of cardiac defect and/or of particular effects of cardiac defect.

In some embodiments, a change in microbiome composition and/or activity comprises any change in abundance and/or type of one or more types of microbes in a microbiome, and/or of one of more components produced thereby. In some embodiment a change in microbiome composition and/or activity comprises an increase in abundance of one or more types of microbes in a microbiome, or of one or more components produced thereby. Alternatively or additionally, in some embodiments, a change in microbiome composition and/or activity comprises a decrease in abundance of one or more types of microbes in a microbiome, and/or of one or more components produced thereby. In some embodiments, a change in microbiome composition and/or activity comprises an increase in abundance of one or more types of microbes, and/or of component(s) produced thereby, and also a decrease in abundance of one or more types of microbes in a microbiome, and/or of component(s) produced thereby.

In accordance with the present invention, microbiome changes that correlate with extent and/or degree of cardiac defect are identified, characterized, and/or detected. In some embodiments, analysis of such changes involves controlling for and/or subtracting out effects of one or more other alterations in microbiome composition and/or activity.

Microbiome composition and/or activity can be detectably altered by events external or internal to a host organism. For example, oral ingestion of antibiotics by individuals can dramatically alter composition and/or activity of their gastrointestinal microbiomes.

In some embodiments a change in microbiome composition and/or activity occurs in response to disease in a host organism. In some embodiments a change in microbiome composition and/or activity occurs in response to infection of a host organism with pathogenic bacteria. In some embodiments a change in microbiome composition and/or activity occurs in response to a change in diet of a host organism. In some embodiments a change in microbiome composition and/or activity occurs in response to a change in water source of a host organism. In some embodiments a change in microbiome composition and/or activity occurs in response to a change in environment of a host organism, for example a person may move to a new city or country. In some embodiments a change in microbiome composition and/or activity occurs in response to a change in personal hygiene habits of a host organism. In some embodiments a change in microbiome composition and/or activity occurs in response to a change in weight of a host organism. In some embodiments a change in microbiome composition and/or activity occurs in response to a change in age of a host organism. In some embodiments a change in microbiome composition and/or activity occurs in response to a change in chemical exposure of a host organism.

In some embodiments a change in microbiome composition and/or activity occurs in response to exposure to microbiome altering agents.

### Microbial Signature

The present invention encompasses the recognition that microbial signatures can be relied upon as proxy for microbiome composition and/or activity. Microbial signatures comprise data points that are indicators of microbiome composition and/or activity. Thus, according to the present invention, changes in microbiomes can be detected and/or analyzed through detection of one or more features of microbial signatures.

In some embodiments, a microbial signature includes information relating to absolute amount of one or more types of microbes, and/or products thereof. In some embodiments, a microbial signature includes information relating to relative amounts of one or more types of microbes and/or products thereof.

In some embodiments, a microbial signature includes information relating to presence, level, and/or activity of at least one type of microbes. In some embodiments, a microbial signature includes information relating to presence, level, and/or activity of between one and 10 types of microbes. In some embodiments, a microbial signature includes information relating to presence, level, and/or activity of between one and 100 types of microbes. In some embodiments, a microbial signature includes information relating to presence, level, and/or activity of between one and 1000 or more types of microbes. In some embodiments, a microbial signature includes information relating to presence, level, and/or activity of substantially all types of microbes within a microbiome.

In some embodiments, a microbial signature comprises a level or set of levels of one or more types of microbes or components or products thereof. In some embodiments, a microbial signature comprises a level or set of levels of one or more DNA sequences. In some embodiments, a microbial signature comprises a level or set of levels of one or more 16S rRNA gene sequences. In some embodiments, a microbial signature comprises a level or set of levels of one or more 18S rRNA gene sequences. In some embodiments, a microbial signature comprises a level or set of levels of one or more RNA transcripts. In some embodiments, a microbial signature comprises a level or set of levels of one or more polypeptides. In some embodiments, a microbial signature comprises a level or set of levels of one or more microbial metabolites.

16S and 18S rRNA gene sequences encode small subunit components of prokaryotic and eukaryotic ribsosomes respectively. rRNA genes are particularly useful in distinguishing between types of microbes because, although sequences of these genes differs between microbial species, the genes have highly conserved regions for primer binding. This specificity between conserved primer binding regions allows the rRNA genes of many different types of microbes to be amplified with a single set of primers and then to be distinguished by amplified sequences.

In methods in accordance with the present invention, a microbial signature is obtained and/or determined using a microbiota sample. A microbiota sample comprises a sample of microbes and or components or products thereof from a microbiome.

In some embodiments, a microbiota sample is collected by any means that allows recovery of microbes or components or products thereof of a microbiome and is appropriate to the relevant microbiome source. For example, where the microbiota sample of the gastrointestinal tract is obtained from a fecal sample.

### Quantifying Microbial Levels

In methods in accordance with the present invention, a microbial signature is obtained and/or determined by quantifying microbial levels. Methods of quantifying levels of microbes of various types are described herein.

In some embodiments, determining a level or set of levels of one or more types of microbes or components or products thereof comprises determining a level or set of levels of one or more DNA sequences. In some embodiments, one or more DNA sequences comprises any DNA sequence that can be used to differentiate between different microbial types. In certain embodiments, one or more DNA sequences comprises 16S rRNA gene sequences. In certain embodiments, one or more DNA sequences comprises 18S rRNA gene sequences. In some embodiments, 1, 2, 3, 4, 5, 10, 15, 20, 25, 50, 100, 1,000, 5,000 or more sequences are amplified.

In some embodiments, a microbiota sample is directly assayed for a level or set of levels of one or more DNA sequences. In some embodiments, DNA is isolated from a microbiota sample and isolated DNA is assayed for a level or set of levels of one or more DNA sequences. Methods of isolating microbial DNA are well known in the art. Examples include but are not limited to phenol-chloroform extraction and a wide variety of commercially available kits, including QIAamp DNA Stool Mini Kit (Qiagen, Valencia, Calif.).

In some embodiments, a level or set of levels of one or more DNA sequences is determined by amplifying DNA sequences using PCR (e.g., standard PCR, semi-quantitative, or quantitative PCR). In some embodiments, a level or set of levels of one or more DNA sequences is determined by amplifying DNA sequences using quantitative PCR. These and other basic DNA amplification procedures are well known to practitioners in the art and are described in Ausebel et al. (Ausubel F M, Brent R, Kingston R E, Moore D D, Seidman J G, Smith J A, Struhl K (eds). 1998. Current Protocols in Molecular Biology. Wiley: New York).

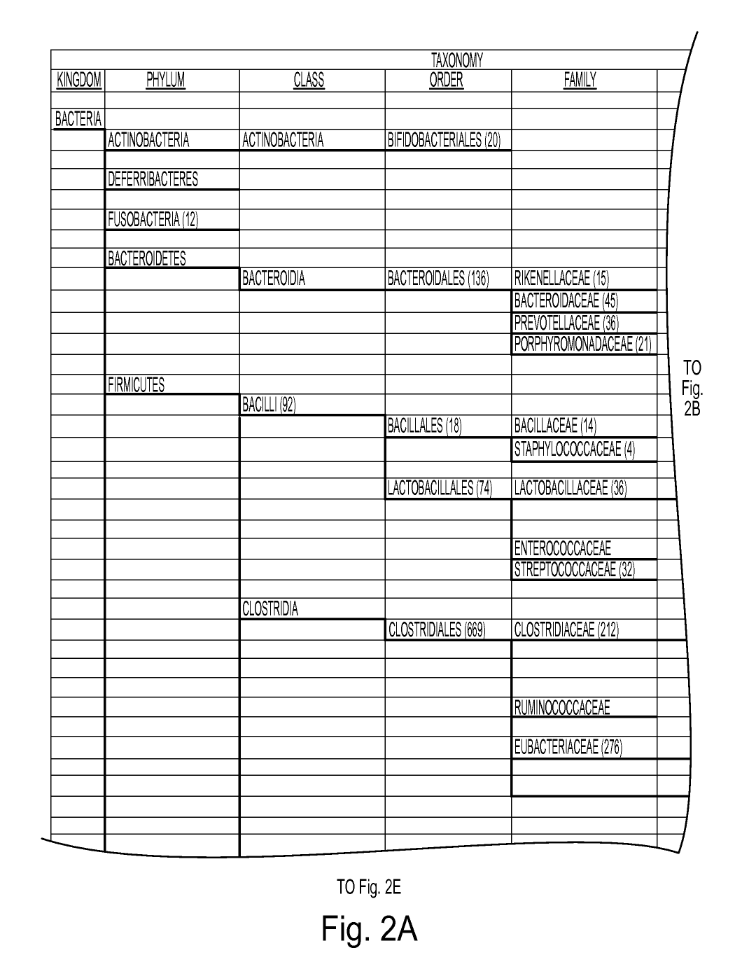

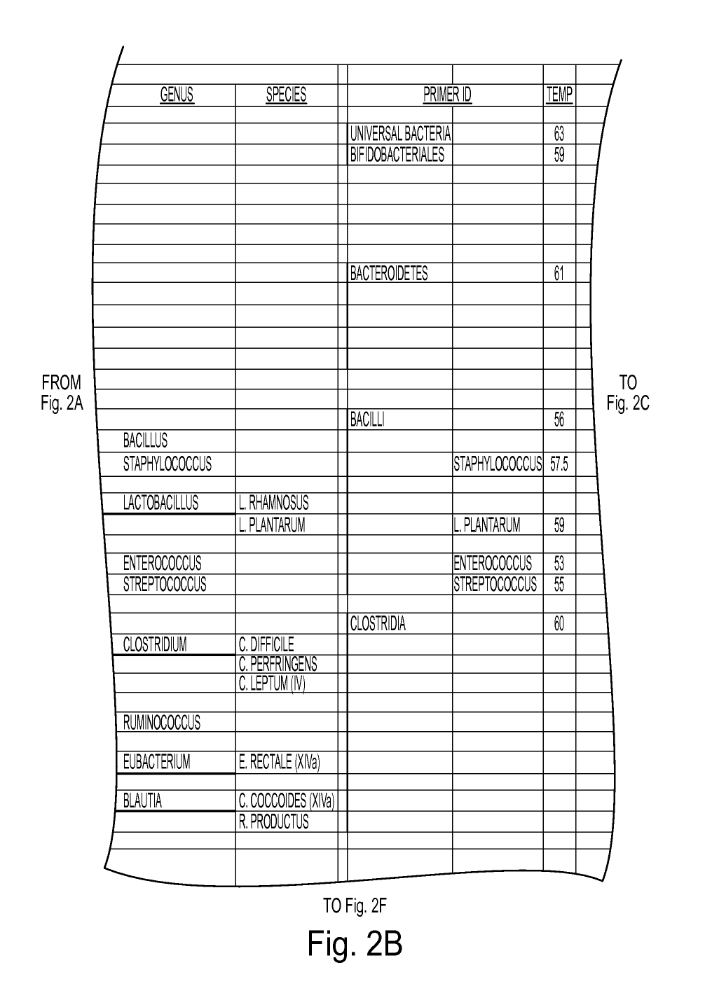

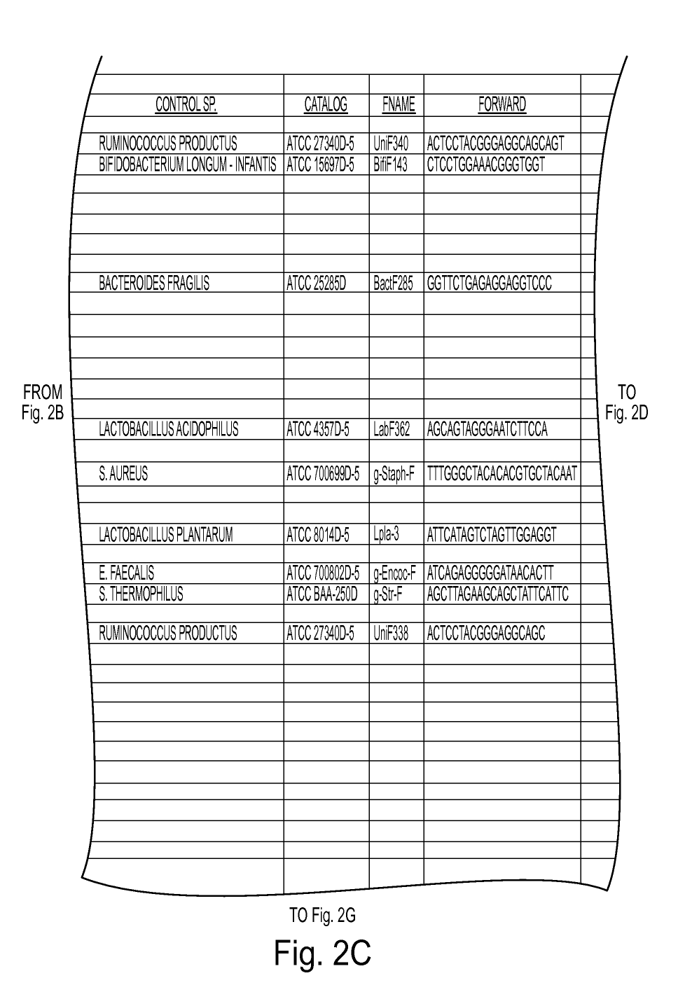

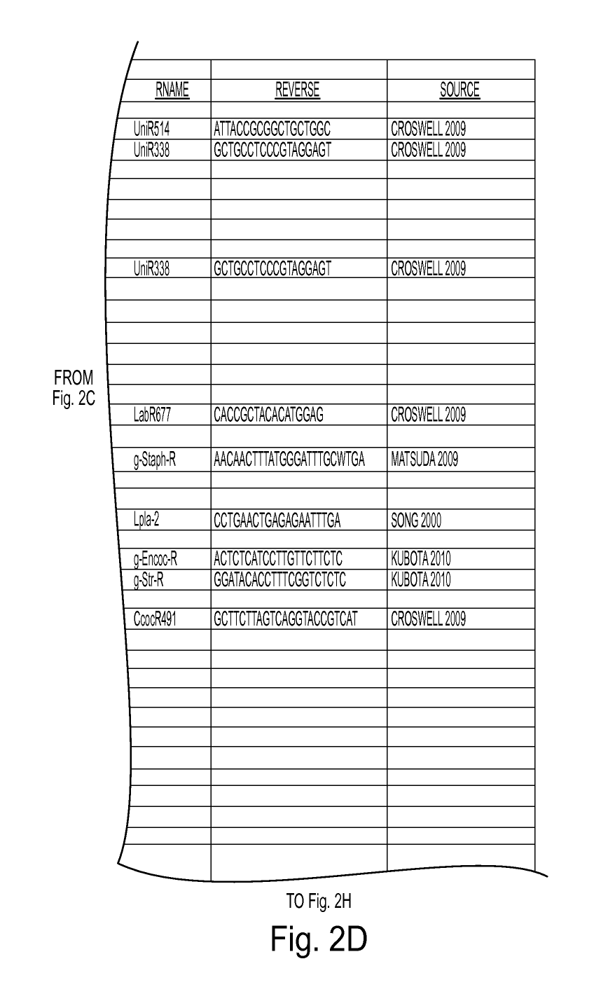

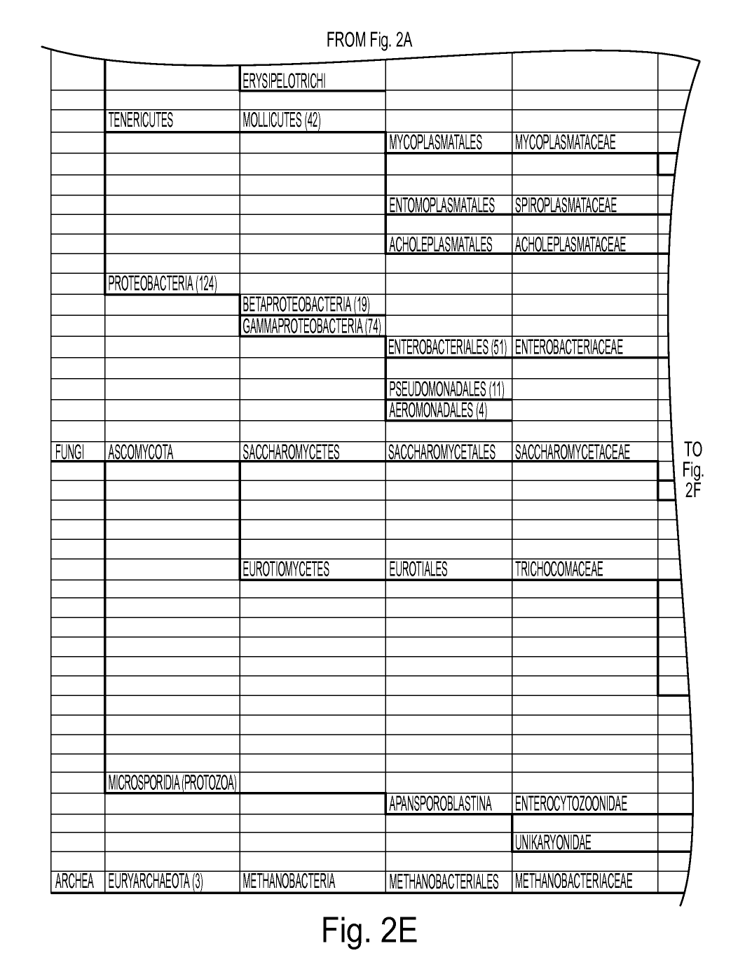

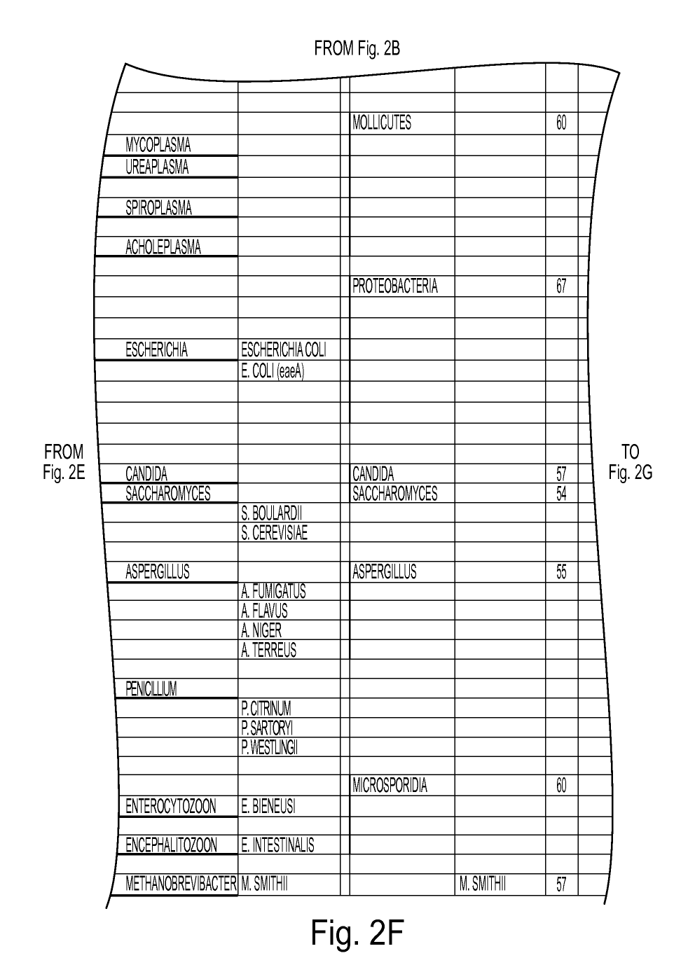

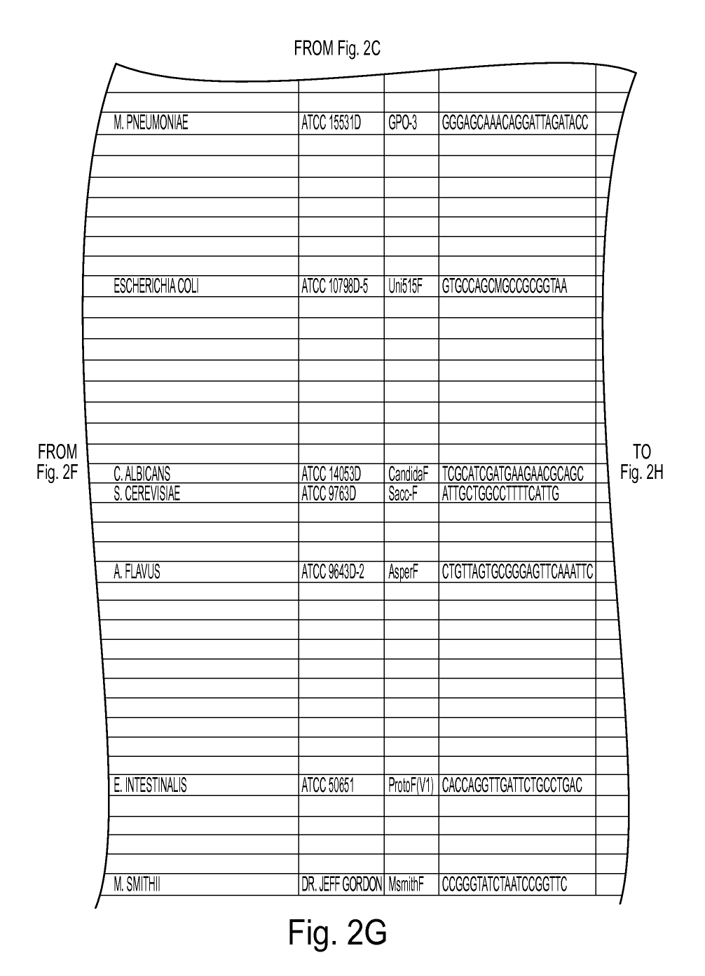

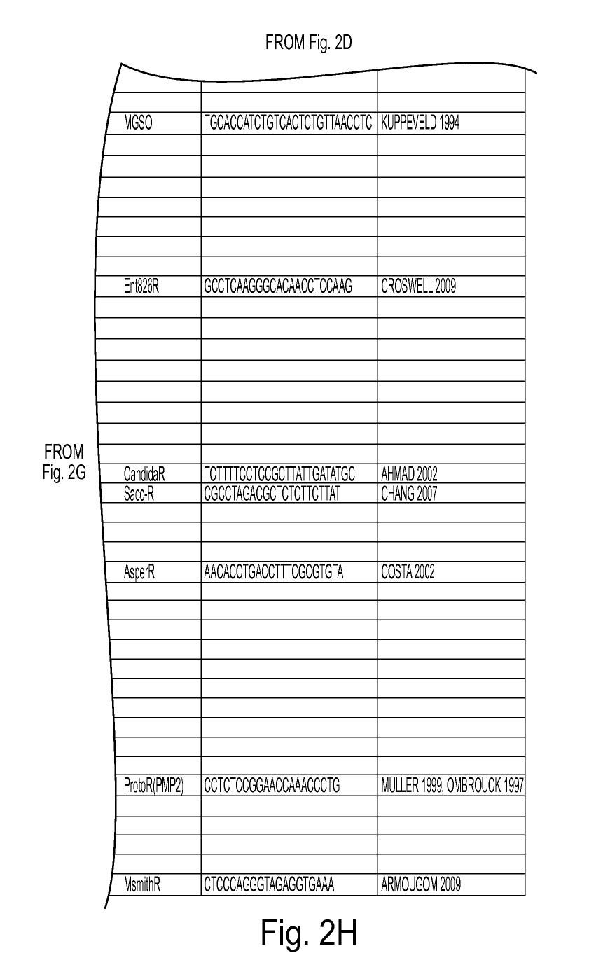

In some embodiments, DNA sequences are amplified using primers specific for one or more sequence that differentiate(s) individual microbial types from other, different microbial types. In some embodiments, 16S rRNA gene sequences or fragments thereof are amplified using primers specific for 16S rRNA gene sequences. In some embodiments, 18S DNA sequences are amplified using primers specific for 18S DNA sequences. In some embodiments, 16S rRNA gene sequences are amplified using primer sequences as shown in FIG. 2.

In some embodiments, a level or set of levels of one or more 16S rRNA gene sequences is determined using phylochip technology. Use of phylochips is well known in the art and is described in Hazen et al. (“Deep-sea oil plume enriches indigenous oil-degrading bacteria.” Science, 330, 204-208, 2010), the entirety of which is incorporated by reference. Briefly, 16S rRNA genes sequences are amplified and labeled from DNA extracted from a microbiota sample. Amplified DNA is then hybridized to an array containing probes for microbial 16S rRNA genes. Level of binding to each probe is then quantified providing a sample level of microbial type corresponding to 16S rRNA gene sequence probed. In some embodiments, phylochip analysis is performed by a commercial vendor. Examples include but are not limited to Second Genome Inc. (San Francisco, Calif.).

In some embodiments, determining a level or set of levels of one or more types of microbes or components or products thereof comprises determining a level or set of levels of one or more microbial RNA molecules (e.g., transcripts). Methods of quantifying levels of RNA transcripts are well known in the art and include but are not limited to northern analysis, semi-quantitative reverse transcriptase PCR, quantitative reverse transcriptase PCR, and microarray analysis. These and other basic RNA transcript detection procedures are described in Ausebel et al.

In some embodiments, determining a level or set of levels of one or more types of microbes or components or products thereof comprises determining a level or set of levels of one or more microbial polypeptides. Methods of quantifying polypeptide levels are well known in the art and include but are not limited to western analysis and mass spectrometry. These and all other basic polypeptide detection procedures are described in Ausebel et al. In some embodiments, determining a level or set of levels of one or more types of microbes or components or products thereof comprises determining a level or set of levels of one or more microbial metabolites. In some embodiments, levels of metabolites are determined by mass spectrometry. In some embodiments, levels of metabolites are determined by nuclear magnetic resonance spectroscopy. In some embodiments, levels of metabolites are determined by enzyme-linked immunosorbent assay (ELISA). In some embodiments, levels of metabolites are determined by colorimetry. In some embodiments, levels of metabolites are determined by spectrophotometry.

Microbial Signatures that Correlate with Cardiac Defect

The present invention encompasses the recognition that changes in microbial signature can be relied upon as proxy for changes in microbiome composition and/or activity. Thus, specific changes in a microbiome to be detected and/or analyzed will contribute to features of a microbial signature. In certain embodiments, the present invention is drawn to methods for defining microbial signatures indicative of risk of cardiac defect and/or of particular effects of cardiac defect by identifying those components of the microbiome that are affected by cardiac defect.

In some embodiments, defining a microbial signature that correlates with a feature of incidence and/or risk of cardiac defect comprises any method that allows identification of types of microbes or components or products thereof that differ between microbiomes of individuals who do and do not suffer from or who have and have not suffered from cardiac defects or that define or classify microbiomes of individuals who suffer from or have suffered from cardiac defects. In some embodiments, defining a microbial signature that correlates with a feature of incidence and/or risk of cardiac defect comprises determining a first set of levels of one or more types of microbes or components or products thereof in a first collection of microbiota samples, where each microbiota sample in the first collection of microbiota samples shares a common feature of incidence and/or risk of cardiac defect; determining a second set of levels of the one or more types of microbes or components or products thereof in a second collection of microbiota samples, which second collection of microbiota samples does not share the common feature of incidence and/or risk of cardiac defect but is otherwise comparable to the first set of microbiota samples; and identifying a microbial signature comprising levels within the first or second set that correlate with presence or absence of the common feature of incidence and/or risk of cardiac defect.

In some embodiments, a collection of microbiota samples comprises at least one microbiota sample. In some embodiments a microbiota sample comprises 1, 2, 3, 5, 10, 15, 20, 25, 30, 35, 40, 45, 50, 100, or 1,000 or more samples.

In some embodiments, the first and second collections of microbiota samples are any two collections of microbiota samples that differ in a feature of incidence and/or risk of cardiac defect but are otherwise comparable. In some embodiments, the first and second collections of microbiota samples are obtained from different host organisms. In some embodiments, the first and second collections of microbiota samples are obtained at from a same collection of hosts at different times.

In some embodiments, a feature of incidence and/or risk of cardiac defect comprises any feature of incidence and/or risk of cardiac defect that allows microbiota samples from host organisms sharing that feature to be distinguished from microbiota samples from host organisms not sharing that feature by methods described herein.

In some embodiments, a feature of incidence and/or risk of cardiac defect comprises incidence of cardiac defect in host organisms from which samples are obtained. In some embodiments, incidence of cardiac defect comprises incidence of any cardiac defect. In some embodiments, incidence of cardiac defect comprises atherosclerosis. In some embodiments, incidence of cardiac defect comprises suffering from coronary heart disease. In some embodiments, incidence of cardiac defect comprises having suffered from myocardial infarction. In some embodiments, incidence of cardiac defect comprises having suffered from a single myocardial infarction event. In some embodiments, incidence of cardiac defect comprises having suffered from 2, 3, 4, 5, 6, 7, 8, 9, 10 or more myocardial infarction events.

In some embodiments, a feature of incidence and/or risk of cardiac defect comprises a feature of incidence of myocardial infarction in host organisms from which samples are obtained. In some embodiments, a feature of incidence of myocardial infarction comprises medical conditions resulting from myocardial infarction. In some embodiments, a feature of incidence of myocardial infarction comprises aneurysms. In some embodiments, a feature of incidence of myocardial infarction comprises pericarditis. In some embodiments, a feature of incidence of myocardial infarction comprises congestive heart failure. In some embodiments, a feature of incidence of myocardial infarction comprises angina. In some embodiments, a feature of incidence of myocardial infarction comprises oedema. In some embodiments, a feature of incidence of myocardial infarction comprises depression. In some embodiments, a feature of incidence of myocardial infarction comprises loss of sex drive or erectile dysfunction. In some embodiments, a feature of incidence of myocardial infarction comprises an enlarged heart.

In some embodiments, a feature of incidence and/or risk of cardiac defect comprises a feature of risk of cardiac defect in host organisms from which samples are obtained. In some embodiments, a feature of risk of cardiac defect comprises any feature of risk cardiac defect that allows microbiota samples from host organisms sharing that feature to be distinguished from microbiota samples from host organisms not sharing that feature by methods described herein. In some embodiments a feature of risk of cardiac defect comprises a risk factor for cardiac defect. In some embodiments a feature of risk of cardiac defect comprises a risk factor for atherosclerosis and/or coronary heart disease. In some embodiments a feature of risk of cardiac defect comprises a risk factor for myocardial infarction.

In some embodiments, a feature of risk of cardiac defect comprises altered susceptibility to ischemia/reperfusion injury in host organisms from which a first set of microbiota samples is obtained relative to host organisms of a second set. In some embodiments, altered susceptibility to ischemia/reperfusion injury comprises a genetic mutation or genetic background known to affect susceptibility to ischemia/reperfusion injury. In some embodiments, a genetic background known to affect susceptibility to ischemia/reperfusion injury comprises mutations in cytochrome p450. In some embodiments, altered susceptibility to ischemia/reperfusion injury comprises exposure to a microbiome-altering agent known to affect susceptibility to ischemia/reperfusion injury.

In some embodiments, altered susceptibility to ischemia/reperfusion injury comprises an altered size of myocardial infarct in host organisms from which a first set of microbiota samples is obtained relative to infarct size in host organisms of a second set. In some embodiments, an altered size of myocardial infarct comprises an increase in myocardial infarct size of between 0 and 1000%. In some embodiments, an altered size of myocardial infarct comprises an increase in myocardial infarct size of between 1 and 100%. In some embodiments, an altered size of myocardial infarct comprises an increase in myocardial infarct size of between 10 and 50%. In some embodiments, an altered size of myocardial infarct comprises a decrease in myocardial infarct size of between 0 and 1000%. In some embodiments, an altered size of myocardial infarct comprises a decrease in myocardial infarct size of between 1 and 100%. In some embodiments, an altered size of myocardial infarct comprises a decrease in myocardial infarct size of between 10 and 50%

In some embodiments, altered susceptibility to ischemia/reperfusion injury comprises altered cardiac output in host organisms from which a first set of microbiota samples is obtained relative to cardiac output in host organisms of a second set. In some embodiments, altered cardiac output comprises altered LVDP. In some embodiments, altered LVDP comprises an increase in LVDP of between 0 and 1000%. In some embodiments, altered LVDP comprises an increase in LVDP of between 1 and 100%. In some embodiments, altered LVDP comprises an increase in LVDP of between 10 and 50%. In some embodiments, altered LVDP comprises a decrease in LVDP of between 0 and 1000%. In some embodiments, altered LVDP comprises a decrease in LVDP of between 1 and 100%. In some embodiments, altered LVDP comprises a decrease in LVDP of between 10 and 50%.

In some embodiments, identifying a microbial signature comprises any means that allows a signature correlated with a feature of cardiac defect to be identified. In some embodiments, identifying a microbial signature comprises identifying one or more levels in a first set of levels in the first collection of microbiota samples that are increased and/or decreased when compared to the second set of levels of the second collection of microbiota samples.

### Uses

Assessing Incidence and/or Risk of Cardiac Defect

The present invention encompasses the recognition that changes in microbial signature can be relied upon as a diagnostic tool to identify and characterize incidence and/or risk of cardiac defect. As described herein, cardiac defects are a major cause of morbidity and mortality worldwide. As such, there is a constant need for more accurate tests for assessing risk of cardiac defect and/or of particular effects of cardiac defect.

In some embodiments, the current invention provides methods of identifying and/or characterizing incidence and/or risk of cardiac defect comprising providing a reference microbial signature that correlates with extent or degree of cardiac defect and determining a microbial signature present in a microbiota sample from an individual whose incidence and/or risk of cardiac defect is to be identified or characterized.

In some embodiments, an individual comprises any individual suffering from or at risk for cardiac defect. In some embodiments, the present invention provides methods of monitoring a patient scheduled to receive or having received a cardiac procedure and the individual comprises a patient.

In some embodiments, a reference microbial signature comprises any value that is correlated with a known feature of incidence and/or risk of cardiac defect. In some embodiments, a reference microbial signature comprises a microbial signature obtained from an individual who does not have and has not had cardiac defect. In some embodiments, a reference microbial signature comprises a microbial signature obtained from an individual who has had a known extent or degree of cardiac defect. In some embodiments, a reference microbial signature comprises a microbial signature obtained from an individual who has had one or more myocardial infarctions. In some embodiments, a reference microbial signature comprises a microbial signature obtained from an individual at low risk for cardiac defect relative to the general population. In some embodiments, a reference microbial signature comprises a microbial signature obtained from an individual having no risk factors for cardiac defect. In some embodiments, a reference microbial signature comprises a microbial signature obtained from an individual having one or more known risk factors for cardiac defect. In some embodiments, a reference microbial signature comprises a microbial signature from an individual who is comparable to the individual whose incidence and/or risk of cardiac defect is to be identified or characterized. In some embodiments, a reference microbial signature comprises a microbial signature from the individual whose incidence and/or risk of cardiac defect is obtained at a different time. In some embodiments, the different time occurred before development of cardiac defect.

In some embodiments, a reference microbial signature is from a microbiota sample of an individual whose incidence and/or risk of cardiac defect is to be identified. In some embodiments, a reference microbial signature comprises a level and/or activity one or more microbes, wherein the level and/or activity of the one or more microbes remains substantially unchanged in response to incidence and/or risk of cardiac defect.

In some embodiments, the current invention provides methods of identifying and/or characterizing incidence and/or risk of cardiac defect comprising providing a reference microbial signature that correlates with extent or degree of cardiac defect, determining a microbial signature present in a microbiota sample from an individual whose incidence and/or risk of cardiac defect is to be identified or characterized and further comprises comparing the microbial signature present in a microbiota sample from an individual whose incidence and/or risk of cardiac defect is to be identified or characterized with the reference microbial signature. In some embodiments, comparing a microbial signature in a microbiota sample from an individual whose incidence and/or risk of cardiac defect is to be identified or characterized with the reference microbial signature comprises comparing microbial signatures obtained from two separate individuals. In some embodiments, comparing microbial signatures comprises comparing microbial signatures obtained from the same individual at separate time points. In some embodiments, comparing microbial signatures comprises comparing microbial signatures of the same microbial sample. In some embodiments, comparing microbial signatures comprises comparing relative levels and/or activities of two or more microbes. In some embodiments, comparing microbial signatures comprises comparing relative levels and/or activities of two or more microbes, wherein at least one first microbe (i.e., level and/or activity of at least one first microbe) remains substantially constant. In some such embodiments, comparing microbial signatures comprises comparing relative levels and/or activities of two or more microbes, wherein at least one second microbe changes.

### Treatment

It is well known that the microbiome of a host plays a significant role in their health and that changes to a hosts microbiome can alter their health status. The present invention encompasses the recognition that microbiomes of an individual can be altered in ways that affect their incidence and/or risk of cardiac defect.

In some embodiments, the current invention provides methods of treating or reducing risk for cardiac defect in an individual by altering the microbiome of the individual, the methods comprising steps of administering to an individual suffering from or susceptible to cardiac defect a composition, such that the individual's microbiome is altered in a manner that correlates with altered severity of or risk for cardiac defect.

In some embodiments, administering comprises any means of administering an effective (e.g., therapeutically effective) or otherwise desirable amount of a composition to an individual. In some embodiments, administering a composition comprises administration by any route, including for example parenteral and non-parenteral routes of administration. Parenteral routes include, e.g., intraarterial, intracerebroventricular, intracranial, intramuscular, intraperitoneal, intrapleural, intraportal, intraspinal, intrathecal, intravenous, subcutaneous, or other routes of injection. Non-parenteral routes include, e.g., buccal, nasal, ocular, oral, pulmonary, rectal, transdermal, or vaginal. Administration may also be by continuous infusion, local administration, sustained release from implants (gels, membranes or the like), and/or intravenous injection.

In some embodiments, altered severity of or risk for cardiac defect comprises any change in severity of or risk for cardiac defect. In some embodiments, altered severity of or risk for cardiac defect comprises a change in levels of agents known to affect severity of or risk for cardiac defect. In some embodiments, an agent known to affect severity of or risk for cardiac defect comprises leptin. In some embodiments, agents known to affect severity of or risk for cardiac defect comprises microbial metabolites. In some embodiments, microbial metabolites comprise 3-(4-hydroxyphenyl)lactate, 3-indoxyl sulfate, 3-phenylpropionate, 4-hydroxyphenylpyruvate, cinnamate, indoleacetate, indolepropionate, kynurenine, p-cresol sulfate, phenol sulfate, phenylacetate, phenylacetylglycine, phenyllactate, or combinations thereof.

In some embodiments, compositions in accordance with the present invention are those that alter an individual's microbiome in a manner that correlates with severity of or risk for a cardiac defect. In some embodiments, compositions in accordance with the present invention are those that, when administered, alter an individual's microbiome in a manner and/or to a state or signature that correlates with reduced severity of or risk for a cardiac defect. In some embodiments, compositions comprise a microbiome altering agent, as described above.

In some embodiments, a composition is administered in an amount and/or according to a dosing regimen that is correlated with a particular desired outcome (e.g., with a particular change in microbiome composition and/or signature that correlates with an outcome of interest). In some embodiments, the desired outcome is alteration (e.g., reduction) in severity of or risk for cardiac defect, as described above.

Particular doses or amounts to be administered in accordance with the present invention may vary, for example, depending on the nature and/or extent of the desired outcome, on particulars of route and/or timing of administration, and/or on one or more characteristics (e.g., weight, age, personal history, genetic characteristic, lifestyle parameter, severity of cardiac defect and/or level of risk of cardiac defect, etc., or combinations thereof). Such doses or amounts can be determined by those of ordinary skill. In some embodiments, an appropriate dose or amount is determined in accordance with standard clinical techniques. Alternatively or additionally, in some embodiments, an appropriate dose or amount is determined through use of one or more in vitro or in vivo assays to help identify desirable or optimal dosage ranges or amounts to be administered.

In some particular embodiments, appropriate doses or amounts to be administered may be extrapolated from dose-response curves derived from in vitro or animal model test systems. The effective dose or amount to be administered for a particular individual can be varied (e.g., increased or decreased) over time, depending on the needs of the individual. In some embodiments, where bacteria are administered, an appropriate dosage comprises at least about 100, 200, 300, 400, 500, 600, 700, 800, 900, 1000 or more bacterial cells. In some embodiments, the present invention encompasses the recognition that greater benefit may be achieved by providing numbers of bacterial cells greater than about 1000 or more (e.g., than about 1500, 2000, 2500, 3000, 35000, 4000, 4500, 5000, 5500, 6000, 7000, 8000, 9000, 10,000, 15,000, 20,000, 25,000, 30,000, 40,000, 50,000, 75,000, 100,000, 200,000, 300,000, 400,000, 500,000, 600,000, 700,000, 800,000, 900,000, 1×10⁶, 2×10⁶, 3×10⁶, 4×10⁶, 5×10⁶, 6×10⁶, 7×10⁶, 8×10⁶, 9×10⁶, 1×10⁷, 1×10⁸, 1×10⁹, 1×10¹⁰, 1×10¹¹, 1×10¹², 1×10¹³ or more bacteria.

In some embodiments, provided compositions include a microbiome altering agent as described herein, together with one or more carriers. In some embodiments, provided compositions comprise one or more pharmaceutically acceptable carriers. In some embodiments, provided compositions comprise one or more edible components. In some embodiments, provided compositions are edible. In some embodiments, provided compositions comprise a microbiome altering agent in a food product, functional food or nutraceutical.

In some embodiments, a food product, functional food or nutraceutical is or comprises a dairy product. In some embodiments, a dairy product is or comprises a yogurt product. In some embodiments, a dairy product is or comprises a milk product. In some embodiments, a dairy product is or comprises a cheese product. In some embodiments, a food product, functional food or nutraceutical is or comprises a juice or other product derived from fruit. In some embodiments, a food product, functional food or nutraceutical is or comprises a product derived from vegetables. In some embodiments, a food product, functional food or nutraceutical is or comprises a grain product, including but not limited to cereal, crackers, bread, and/or oatmeal. In some embodiments, a food product, functional food or nutraceutical is or comprises a rice product. In some embodiments, a food product, functional food or nutraceutical is or comprises a meat product.

In some embodiments, a provided composition is provided as a pharmaceutical formulation. In some embodiments, a pharmaceutical formulation is or comprises a unit dose amount for administration in accordance with a dosing regimen correlated with achievement of the reduced severity or risk of cardiac defect.

In some embodiments, a pharmaceutical formulation comprises a capsule (e.g., a gelatin capsule) or tablet (e.g., a pressed tablet). In some embodiments, a pharmaceutical formulation is or comprises a liquid.

In some embodiments, provided compositions, including those provided as pharmaceutical formulations, comprise a liquid carrier such as but not limited to water, saline, phosphate buffered saline, Ringer's solution, dextrose solution, serum-containing solutions, Hank's solution, other aqueous physiologically balanced solutions, oils, esters and glycols.

In some embodiments, a unit dose is administered in accordance with a dosing regimen correlated with achievement of the reduced severity or risk of cardiac defect. In some embodiments, a dosing regimen comprises administration as a single unit dose. In some embodiments, a dosing regimen comprises administration of multiple unit doses separated from one another by time intervals. Administration at an “interval,” as used herein, indicates that the unit dose is administered periodically (as distinguished from a one-time dose). In some embodiments, unit doses are separated by identical time intervals; in some embodiments, unit doses are separated by different intervals. In some embodiments, unit doses are administered, for example, bimonthly, monthly, twice monthly, triweekly, biweekly, weekly, twice weekly, thrice weekly, daily, twice daily, or every six hours.

As used herein, the term “bimonthly” means administration once per two months (i.e., once every two months); the term “monthly” means administration once per month; the term “triweekly” means administration once per three weeks (i.e., once every three weeks); the term “biweekly” means administration once per two weeks (i.e., once every two weeks); the term “weekly” means administration once per week; and the term “daily” means administration once per day.

### Combination Therapy

In some embodiments, compositions as described herein are administered in combination with one or more other agents or factors that affect or alter one or more aspects of an individual's physiology and/or of an individual's microbiome. For example, in some embodiments, provided compositions are administered in combination with one or more anti-proliferatives (e.g., antibiotics), anti-inflammatories, pain relievers, etc. In some embodiments, provided compositions are administered in combination with one or more known therapeutic agents, and the known therapeutic agent(s) is/are administered according to its standard or approved dosing regimen and/or schedule. In some embodiments, provided compositions are administered in combination with one or more known therapeutic agents, and the known therapeutic agent(s) is/are administered according to a regimen that is altered as compared with its standard or approved dosing regimen and/or schedule. In some embodiments, such an altered regimen differs from the standard or approved dosing regimen in that one or more unit doses is altered (e.g., reduced or increased) in amount, and/or in that dosing is altered in frequency (e.g., in that one or more intervals between unit doses is expanded, resulting in lower frequency, or is reduced, resulting in higher frequency).

### EXEMPLIFICATION

### Example 1

### Method for Rat Handling and Antibiotic Treatment

In the following example, methods for handling and antibiotic treating rats are described. Rat handling and use protocols were approved by the Institutional Animal Care and Use Committee at the Medical College of Wisconsin. Male Dahl S rats (200-220 g; Charles River, Wilmington, Mass.) were fed autoclavable laboratory rodent diet 5010 (LabDiet, St. Louis, Mo.) and given water ad libitum for one week prior to antibiotic treatment. Vancomycin, an antibiotic known to alter gastrointestinal microbiota (Croswell, A. et al. “Prolonged impact of antibiotics on intestinal microbial ecology and susceptibility to enteric Salmonella infection.” Infect. Immun. 77, 2741-2753, 2009) was added to drinking water (0.5 g/L).

Rats were anesthetized by intraperitoneal injection of pentobarbital (Nembutal; 50 mg/kg) and euthanized with an overdose of intraperitoneal pentobarbital with a pneumothorax performed. While anesthetized, rats were monitored for anesthetic depth via assessments of the pedal reflex and respiratory rate. Surgical procedures were not continued unless the pedal reflex was lost. In addition, the pedal reflex was monitored every 15-30 min during the procedure, and if detected, the rat was administered additional sodium pentobarbital.

### Example 2

### Assaying Fecal Microbiota Abundance in Rats

In the following example, methods for quantifying microbiota abundance in rat feces are described. Fresh fecal pellets were obtained from each rat prior to (day 0) and at day 6 to 7 post treatment. Pellets were homogenized in 1 ml PBS. 200 μl of homogenate was used for microbial DNA isolation using a QIAamp DNA Stool Mini Kit (Qiagen, Valencia, Calif.). Isolated DNA samples were subjected to quantitative PCR using an iCycler (Bio-Rad, Hercules, Calif.) for microbial population enumeration. PCR reaction mixture consisted of 50% iQ SYBR Green Supermix (Bio-Rad), 0.4 μM forward and reverse primers, and 3.8% template solution in RNase/DNase free water. Primer sets specific for 16s and 18s rRNA genes of particular microbial phylum, class, genus, and species (M. smithii and L. plantarum) along with 20 reaction temperature and reference strains are detailed in FIG. 2 and Table 1.

TABLE 1
16S and 18S Primer Pairs

| | | Primer | |
|---|---|---|---|
| Primer ID | Species | Name | Primer Sequence |
| Universal | Ruminococcus | UniF340 | ACTCCTACGGGAGGCAGCAGT |
| Bacteria | productus | | (SEQ ID NO: 1) |
| Universal | Ruminococcus | UniR514 | ATTACCGCGGCTGCTGGC |
| Bacteria | productus | | (SEQ ID NO: 2) |
| Bifidobacteriales | Bifidobacterium | Infantis | CTCCTGGAAACGGGTGGT |
| | longum | BifiF143 | (SEQ ID NO: 3) |
| Bifidobacteriales | Bifidobacterium | UniR338 | GCTGCCTCCCGTAGGAGT |
| | longum | | (SEQ ID NO: 4) |
| Bacteroidetes | Bacteroides | BactF285 | GGTTCTGAGAGGAGGTCCC |
| | fragilis | | (SEQ ID NO: 5) |
| Bacteroidetes | Bacteroides | UniR338 | GCTGCCTCCCGTAGGAGT |
| | fragilis | | (SEQ ID NO: 6) |
| Bacilli | Lactobacillus | LabF362 | AGCAGTAGGGAATCTTCCA |
| | acidophilus | | (SEQ ID NO: 7) |
| Bacilli | Lactobacillus | LabR677 | CACCGCTACACATGGAG |
| | acidophilus | | (SEQ ID NO: 8) |
| Staphylococcus | S. aureus | g-Staph-F | TTTGGGCTACACACGTG |
| | | | CTACAATGGACAA |
| | | | (SEQ ID NO: 9) |
| Staphylococcus | S. aureus | g-Staph-R | AACAACTTTATGGG |
| | | | ATTTGCWTGA |
| | | | (SEQ ID NO: 10) |
| L. plantarum | Lactobacillus | Lpla-3 | ATTCATAGTCTAGTTGGAGGT |
| | plantarum | | (SEQ ID NO: 11) |
| L. plantarum | Lactobacillus | Lpla-2 | CCTGAACTGAGAGAATTTGA |
| | plantarum | | (SEQ ID NO: 12) |
| Enterococcus | E. faecalis | g-Encoc-F | ATCAGAGGGGGATAACACTT |
| | | | (SEQ ID NO: 13) |
| Enterococcus | E. faecalis | g-Encoc-R | ACTCTCATCCTTGTTCTTCTC |
| | | | (SEQ ID NO: 14) |
| Streptococcus | S. thermophilus | g-Str-F | AGCTTAGAAGCAGCTATTCATTC |
| | | | (SEQ ID NO: 15) |
| Streptococcus | S. thermophilus | g-Str-R | GGATACACCTTTCGGTCTCTC |
| | | | (SEQ ID NO: 16) |
| Clostridia | Ruminococcus | UniF338 | ACTCCTACGGGAGGCAGC |
| | productus | | (SEQ ID NO: 17) |
| Clostridia | Ruminococcus | CcocR491 | GCTTCTTAAGTCAGGTACCGTCAT |
| | productus | | (SEQ ID NO: 18) |
| Mollicutes | M. pneumonia | GPO-3 | GGGAGCAAACAGGA |
| | | | TTAGATACCCT |
| | | | (SEQ ID NO: 19) |
| Mollicutes | M. pneumonia | MGSO | TGCACCATCTGTCACTCT |
| | | | GTTAACCTC |
| | | | (SEQ ID NO: 20) |
| Proteobacteria | Escherichia coli | Uni515F | GTGCCAGCMGCCGCGGTAA |
| | | | (SEQ ID NO: 21) |
| Proteobacteria | Escherichia coli | Ent826R | GCCTCAAGGGCACAACCTCCAAG |
| | | | (SEQ ID NO: 22) |
| Candida | C. albicans | CandidaF | TCGCATCGATGAAGAACGCAGC |
| | | | (SEQ ID NO: 23) |
| Candida | C. albicans | CandidaR | TCTTTTCCTCCGCTTATTGATATGC |
| | | | (SEQ ID NO: 24) |
| Saccharomyces | S. cerevisiae | Sacc-F | ATTGCTGGCCTTTTCATTG |
| | | | (SEQ ID NO: 25) |
| Saccharomyces | S. cerevisiae | Sacc-R | CGCCTAGACGCTCTCTTCTTAT |
| | | | (SEQ ID NO: 26) |
| Aspergillus | A. flavus | AsperF | CTGTTAGTGCGGGAG |
| | | | TTCAAATTCT |
| | | | (SEQ ID NO: 27) |
| Aspergillus | A. flavus | AsperR | AACACCTGACCTTTCGCGTGTA |
| | | | (SEQ ID NO: 28) |
| Microsporidia | E. intestinalis | ProtoF | CACCAGGTTGATTCTGCCTGAC |
| | | (V1) | (SEQ ID NO: 29) |
| Microsporidia | E. intestinalis | ProtoR | CCTCTCCGGAACCAAACCCTG |
| | | (PMP2) | (SEQ ID NO: 30) |
| M. smithii | M. smithii | MsmithF | CCGGGTATCTAATCCGGTTC |
| | | | (SEQ ID NO: 31) |
| M. smithii | M. smithii | MsmithR | CTCCCAGGGTAGAGGTGAAA |
| | | | (SEQ ID NO: 32) |

### Example 3

### Quantification of Myocardial Infarction in Rats

In the following example, methods for assaying myocardial infarction in vitro and in vivo in rats as determined by ischemia/reperfusion studies are described.

An anesthetized rat model was used for in vivo ischemia/reperfusion studies using the general surgical protocol and determination of infarct size (IS) described in “K(ATP) opener-induced delayed cardioprotection: involvement of sarcolemmal and mitochondrial K(ATP) channels, free radicals and MEK½” (Gross, E. et al., J. Mol. Cell. Cardiol. 35, 985-992, 2003), the entirety of which is incorporated herein by reference. Briefly, following anesthesia, a tracheotomy for artificial ventilation was performed, with the left common carotid artery cannulated for blood pressure and heart rate measurements. A thoracotomy was performed at the fifth intercostal space, the pericardium was excised, and a silk ligature was placed distal to the left atrial appendage that spanned to the sternal portion of the left ventricle, which included the left anterior descending coronary artery. Occlusion of the area described [area at risk (AAR)] was created by placing ends of the ligature through a polypropylene tube and fixing the snare to the epicardial surface with a hemostat. After 30 min, the hemostat was released to reperfuse the AAR. Following 2 h of reperfusion, the ligature was again occluded, and the AAR was determined by patent-blue negative staining. The heart was then excised, cross-sectioned into 4 to 5 slices, and separated into normal zone and AAR. Pieces were incubated in 1% 2,3,5-triphenyltetrazolium chloride to determine IS. The heart was then incubated overnight in 10% formaldehyde, and infarcted tissue was dissected from the AAR. IS was expressed as a percentage of the AAR (IS/AAR). Leptin (0.12 μg/kg) was administered intravenously as a bolus at 24 and 12 h before ischemia. Amino acids were administered intravenously as a bolus at 24 and 12 hours before ischemia or dissolved in the drinking water and given 48 hours before ischemia.

For in vitro ischemia/reperfusion studies, hearts were perfused retrogradely, as described previously in Baker, J. et al. (Am. J. Physiol. Heart Circ. Physiol., 2000), the entirety of which is incorporated herein by reference. Briefly, hearts were perfused with modified Krebs-Henseleit buffer (120 mM NaCl, 25 mM NaHCO₃, 4.7 mM KCl, 1.2 mM KH2PO₄, 1.20 mM MgSO₄, 11 mM glucose, and 1.8 mM CaCl₂) bubbled with 95% O₂-5% CO₂ for a 40-minute stabilization period and subjected to 25 minutes of global no-flow ischemia, followed by 180 minutes of reperfusion. Before use, all perfusion fluids were filtered through cellulose acetate membranes with a pore size of 5.0 μm to remove particulate matter. Hearts were kept in temperature-controlled chambers to maintain myocardial temperature at 37° C. A balloon connected to a pressure transducer was inserted into the left ventricle to monitor cardiac function. For some experiments, hearts were stabilized for 25 minutes and then perfused with vancomycin or a mixture of antibiotics for 15 minutes before ischemia/reperfusion. During the initial 40 minute reperfusion period, recovery of mechanical function was measured as left ventricular developed pressure (LVDP) under steady-state conditions and expressed as a percentage of preischemic LVDP. At the end of the 3 hour reperfusion period, hearts were processed and stained with 2,3,5-triphenyltetrazolium chloride dye for IS determination.

### Example 4

### Effect of Vancomycin and Probiotics on Intestinal Microbiota and Myocardial Infarction

In the following example, the effects of treatment of rats with vancomycin and probiotics on intestinal microbiota and myocardial infarction are assessed. The present example is described in the publication by the inventor titled “Intestinal Microbiota determine severity of myocardial infarction in rats,” (FASEB, in press, published on-line 2012) the entirety of which is incorporated herein by reference. Vancomycin, a minimally absorbable antibiotic, was added to the drinking water as described in example 1 as a tool to alter intestinal microbiota composition of Dahl S rats. Rats received antibiotic-supplemented drinking water for up to 7 d. Microbial populations present in feces were monitored by 16S/18S rRNA quantitative-PCR as described in example 2. Primers used uniquely targeted 16S rRNA genes of each eubacteria and archaea taxon and unique 18S rRNA genes of each fungal taxon. The results presented herein indicate that vancomycin reduced total bacterial numbers but had species specific varying effects on microbial composition (FIG. 3).

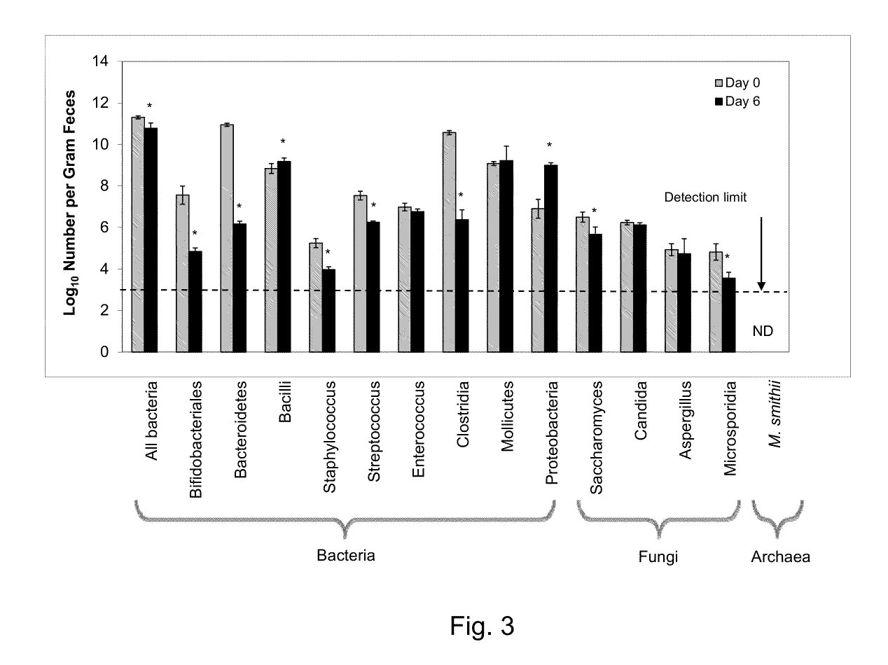

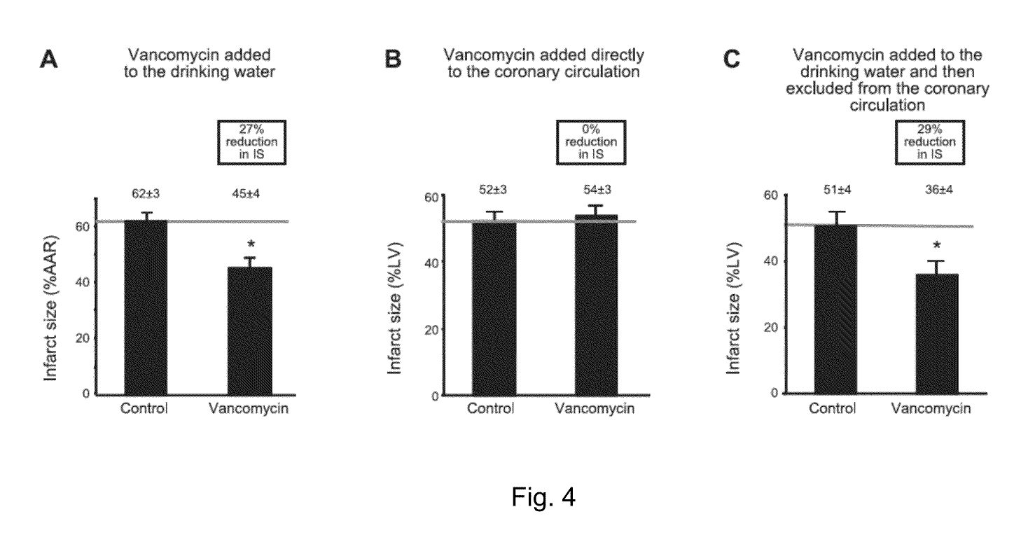

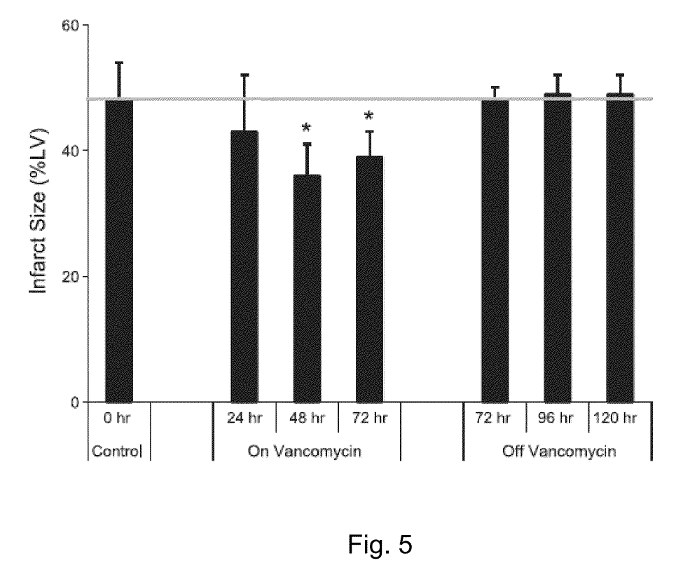

Dahl S rats are an established model of increased susceptibility to injury from myocardial ischemia/reperfusion (Baker, J. et al. “Resistance to myocardial ischemia in five rat strains: is there a genetic component of cardioprotection?” Am. J. Physiol. Heart Circ. Physiol. 278, H1395-H1400, 2000, Shi, Y. et al. “Increased resistance to myocardial ischemia in the Brown Norway vs. Dahl S rat: role of nitric oxide synthase and Hsp90.” J. Mol. Cell. Cardiol. 38(4):625-635, 2005). Vancomycin added to drinking water for 7 d as described in example 1 decreased susceptibility of hearts to injury in an in vivo model of regional myocardial ischemia/reperfusion as described in example 3, manifest by a reduction in myocardial IS of ˜27% (FIG. 4A). A minimum treatment time of 48 h with vancomycin was needed to decrease IS. A return to control values for myocardial IS at 72 h following discontinuation of vancomycin was observed (FIG. 5). To determine whether this decrease in IS was a direct effect of antibiotic present in the coronary vasculature, blood levels of vancomycin were measured. The concentration of vancomycin in blood was below detection limits of the assay used (1 μM). When vancomycin was added directly to coronary perfusate at a concentration of 1 μM in an in vitro model of myocardial ischemia/reperfusion in rats, as described in example 3, there was no reduction in IS (FIG. 4B). To determine whether the decrease in IS was indirect, vancomycin was added to drinking water and then excluded from coronary perfusate before ischemia/reperfusion, using an in vitro model of myocardial infarction, as described in example 3. Vancomycin decreased IS by 29% (FIG. 4C). The results herein demonstrate vancomycin reduces IS in Dahl S rats despite absence of antibiotic in coronary perfusate at ischemia/reperfusion. These data suggest that a direct effect of antibiotic on hearts is not responsible for decreased IS. The extent of reduction in IS using in vivo and in vitro models of myocardial ischemia/reperfusion was comparable (FIG. 4A, C)

The concentration of cytokines in blood of rats treated with vancomycin was quantified to identify antibiotic-induced changes. Blood samples were obtained for cytokine analysis before antibiotic treatment (d 0) and at d 6 of antibiotic treatment. Blood samples were kept on ice for 30 min and then centrifuged at 1000 g for 10 min at 4° C. to obtain plasma. Plasma samples were then analyzed to determine the concentration of 23 cytokines (Eve Technologies, Calgary, AB, Canada). Antibiotic was administered continuously via drinking water as described in example 1. Of 23 cytokines measured, 11 were reliably quantified. Of these, only leptin was significantly different between control and treatment groups (FIG. 6A). Vancomycin decreased circulating leptin by 38±4%. To determine whether decreased leptin levels were associated with a reduction in IS, vancomycin-treated rats were administered leptin (0.12 μg/kg i.v.) at 24 and 12 h before ischemia/reperfusion in vitro as described in example 3. This dose was selected to reconstitute leptin concentrations in circulation. Leptin abolished the decrease in IS and increase in recovery of LVDP conferred by vancomycin treatment (FIG. 6B). Leptin treatment in the absence of vancomycin pretreatment had no effect. The present disclosure therefore indicates that the decrease in IS and increase in recovery of LVDP seen with vancomycin administration can be counteracted by leptin administration.

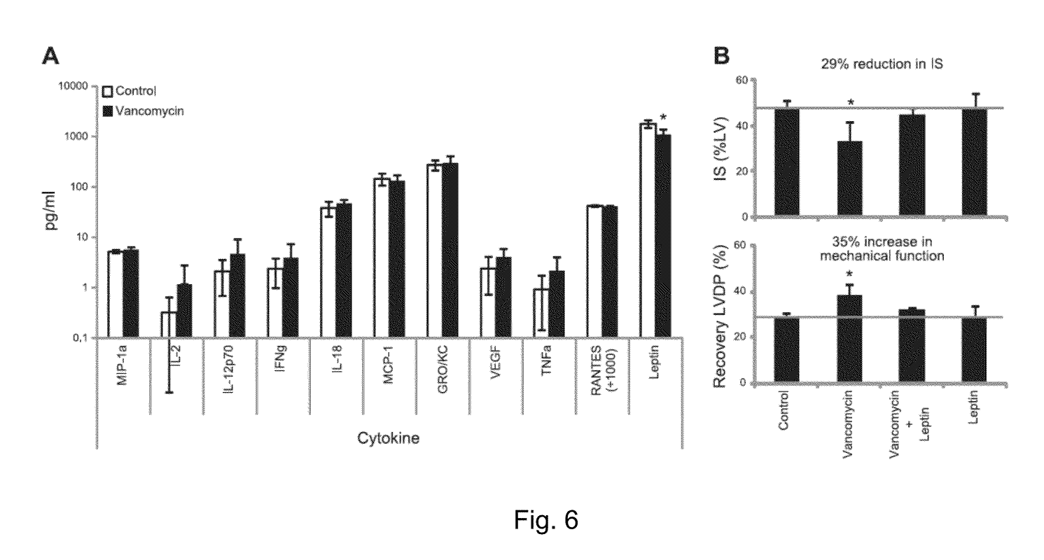

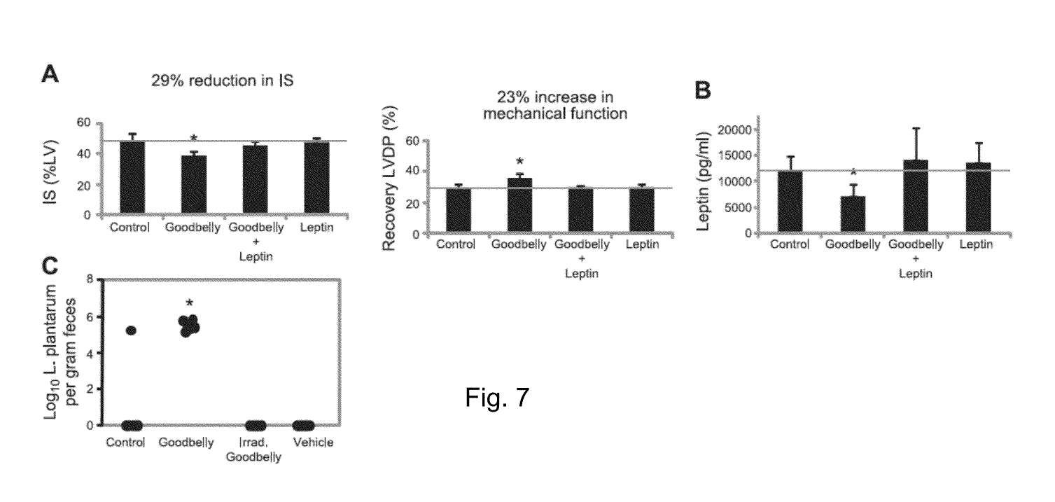

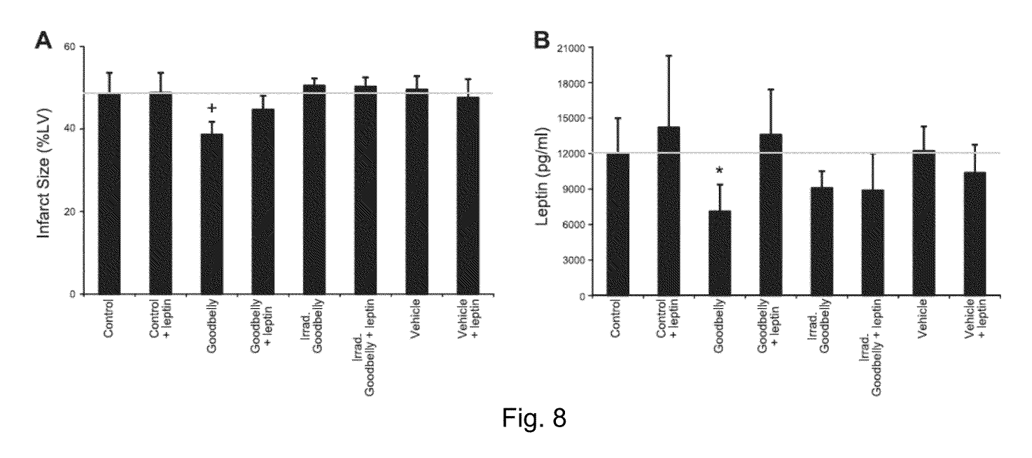

L. plantarum, a probiotic, lowers leptin levels by 37% in smokers (Naruszewicz, M. et al. “Effect of Lactobacillus plantarum 299v on cardiovascular disease risk factors in smokers.” Am. J. Clin. Nutr. 76, 1249-1255, 2002) and in mice fed a high-fat diet by 38% (Takemura, N., et al. “Lactobacillus plantarum strain No. 14 reduces adipocyte size in mice fed high-fat diet.” Exp. Biol. Med. 235, 849-856, 2010). To determine whether probiotic juice containing two probiotic bacteria, L. plantarum (Lp299v) and Bifidobacterium lactis(Bi-07), can reduce leptin levels and severity of myocardial infarction, rats were fed probiotic juice containing L. plantarum (Lp299v) and Bifidobacterium lactis(Bi-07) for 14 d (hereinafter referred to as “probiotic juice”). Probiotic juice was administered 1×/d in addition to drinking water. Each liter bottle was stored at 4° C. until provided to rats (15 ml/rat/d). Forty-five milliliters of probiotic juice or vehicle was portioned into a small bottle with a lick spout and made available to each cage of 3 rats. Rats readily consumed probiotic juice within 15 min and were monitored to ensure that none of the rats were excluded from drinking Negative controls for probiotic juice treatment included irradiated probiotic juice (35 kGy; Sterigenics, Gurnee, Ill., USA) and sugar water (water, 92.8 mg/ml glucose, 42.2 μg/ml NaCl, 464 μg/ml KCl, and 4 mg/ml albumin) to control for sugar, salt, and protein content of the probiotic juice. Rats were fed these negative controls in the same quantities as probiotic juice. Ischemia/reperfusion was performed as described in example 3. A 29% decrease in IS was observed in probiotic juice-fed rats (FIG. 7A). Administration of leptin (0.12 μg/kg i.v.) at 24 and 12 h before ischemia/reperfusion abolished probiotic juice-induced cardioprotection (FIG. 7A). γ-Irradiated (35 kGy) probiotic juice, irradiated probiotic juice plus leptin, probiotic juice equivalent vehicle, and vehicle plus leptin had no effect on IS (FIG. 8A). Probiotic juice treatment also decreased leptin levels in blood by 41% (FIG. 7B). Gamma irradiated (35 kGy) probiotic juice, irradiated probiotic juice plus leptin, probiotic juice equivalent vehicle, and vehicle plus leptin had no effect on leptin levels (FIG. 8B). L. plantarum was not detectable in feces of rats fed control, vehicle, and irradiated probiotic juice but was present at 5.8 log₁₀/g feces in probiotic juice-treated rats (FIG. 7). The results presented herein demonstrate that treatment with probiotic juice containing L. plantarum (Lp299v) and Bifidobacterium lactis(Bi-07) decreases IS and leptin levels.

The results of the present disclosure encompass a proof of concept and a mechanistic link between changes in intestinal microbiota and myocardial infarction. To demonstrate the role of intestinal microbiota in predicting severity of myocardial infarction, rats were treated orally with the broad-spectrum antibiotic vancomycin to effectively reduce total microbiota numbers and alter the abundance of individual groups of intestinal microbiota. The cardioprotective effect of vancomycin is indirect as this antibiotic is minimally absorbed into the circulation and, when administered directly into the coronary circulation, had no effect on severity of myocardial infarction. When vancomycin was added to the drinking water and the heart was isolated from the circulatory system, a reduction in IS was still observed. Thus, protection was established within the myocardium by vancomycin treatment and manifest despite the absence of the antibiotic in the circulation before ischemia/reperfusion. Cardioprotection was established within 2 d of treatment and lost after treatment ceased for 3 d. Cytokine levels in the plasma of vancomycin-treated rats were analyzed to identify signaling molecules that might mediate myocardial IS. Leptin levels were decreased 38% by vancomycin treatment. The probiotic juice, which contains both L. plantarum (Lp299v) and B. lactis (Bi-07), also decreased circulating leptin levels similarly to vancomycin and reduced myocardial IS to the same extent as vancomycin. In either case, pretreatment with leptin abolished cardioprotection by both vancomycin and probiotic juice.

In the current state of the art, little is understood of the host-microbiome interactions that influence host cardiovascular functions. The results presented herein encompass a proof of concept demonstrating that a perturbation of intestinal microbiota manifests itself in a host's systemic metabolic phenotype that is capable of affecting severity of myocardial infarction. Metabolites synthesized by the microbiome actively influence host biology, and any dysbiosis in this virtual organ has implications for the host health. L. plantarum, a member of the bacilli taxonomy class of bacteria, is known to reduce the cardiovascular disease biomarkers fibrinogen and LDL-cholesterol in addition to regulating leptin levels in the circulation (Naruszewicz, M. et al. “Effect of Lactobacillus plantarum 299v on cardiovascular disease risk factors in smokers.” Am. J. Clin. Nutr. 76, 1249-1255, 2002). The present disclosure demonstrates that decreased blood leptin concentrations from vancomycin and probiotic juice treatment before ischemia/reperfusion results in increased cardioprotection.

Leptin is a 16-kDa, 167-aa polypeptide synthesized and secreted into the circulation primarily by white adipocytes. The heart is also a site of leptin production and action (Purdham, D. M. et al. “Rat heart is a site of leptin production and action.” Am. J. Physiol. Heart Circ. Physiol. 287, H2877-H2884, 2004). Leptin binding to its receptor activates several intracellular pathways, including JAK/STAT, MAPK, Akt, and mammalian target of rapamycin. Leptin treatment induces cardioprotection by activating JAK/STAT and Akt signaling (Smith, C. C. et al. “Leptin-induced cardioprotection involves JAK/STAT signaling that may be linked to the mitochondrial permeability transition pore.” Am. J. Physiol. Heart Circ. Physiol. 299, H1265-H1270, 2010).

The present disclosure is supportive of the model of myocardial leptin resistance, which suggests that persistent high levels of leptin in the circulation desensitizes the myocardium to leptin signaling (Ren, J. et al. “High-fat diet-induced obesity leads to resistance to leptin-induced cardiomyocyte contractile response.” Obesity 16, 2417-2423, 2008). Conversely, persistent reduction in the level of leptin in the circulation enhances the sensitivity of the myocardium to leptin. Taken together, these results suggest that a probiotic may be able to affect a biphasic protection of the myocardium by reducing the level of leptin in the circulation. As demonstrated by the results herein, decreased leptin levels in the circulation decreases the myocardium's susceptibility to acute injury from ischemia/reperfusion while other studies have showed that reduced leptin signaling through blockade of the leptin receptor results in decreased chronic cardiac hypertrophy (Purdham, D. M. et al, “A neutralizing leptin receptor antibody mitigates hypertrophy and hemodynamic dysfunction in the postinfarcted rat heart.” Am. J. Physiol. Heart Circ. Physiol. 295, H441-H446, 2008). The present disclosure therefore indicates that altering the intestinal microbiota with probiotics to decrease leptin levels in the circulation will be able to mitigate or treat hypertrophy and cardiac remodeling after myocardial infarction.

The results described herein suggest a new approach to prevent or treat myocardial infarction: the use of probiotics to supplement the diet. The identification and administration of additional microbial species capable of controlling leptin in the circulation would be therapeutically beneficial and less disruptive to the intestinal microbiota than broad spectrum antibiotics. The magnitude of cardioprotection with vancomycin and probiotic juice is comparable with that of pharmacologic preconditioning with erythropoietin (39% reduction in IS; Baker, J. E. et al. “Darbepoetin alfa protects the rat heart against infarction: dose-response, phase of action, and mechanisms.” J. Cardiovasc. Pharmacol. 49, 337-345, 2007) and thrombopoietin (34% reduction in IS; Baker, J. E. et al. “Human thrombopoietin reduces myocardial infarct size, apoptosis, and stunning following ischaemia/reperfusion in rats.” Cardiovasc. Res. 77, 44-53, 2008). Pharmacologic preconditioning involves administering a pharmacologic agent, either before, during, or at the onset of reperfusion following sustained ischemia, to confer cardioprotection.

An estimated 1.4 million people in the United States will have a new or recurrent acute myocardial infarction every year, with many survivors experiencing lasting morbidity, progression to heart failure, and death (Roger, V. L. et al. “Heart disease and stroke statistics—2011 update: a report from the American Heart Association.” Circulation 123, e18-e209, 2011). The present disclosure demonstrates a proof-of-concept relationship between intestinal microbiota-derived metabolites and myocardial infarction that provides opportunities for both novel diagnostic tests (fecal microbiota and/or microbial metabolites in feces and/or blood as biomarkers of susceptibility to myocardial infarction) and therapeutic approaches (probiotics, nonabsorbable antimicrobials, and/or microbial metabolites) for the treatment and prevention of myocardial infarction and hypertrophy.

### Example 5

### Mediators of Vancomycin Induced Cardioprotection

In the following example, effects of blocking intracellular signaling pathways on vancomycin induced cardioprotection are described. Receptor binding and activation of pro-survival kinases JAK-2, Akt, p42/44, MAPK and p38 MAPK, as well as activation of ATP-dependent potassium (K_{ATP}) channels are classical mediators of cardioprotection. These mediators are components of intersecting and interdependent signaling pathways in the myocardium that determine severity of myocardial infarction. The role of these mediators in vancomycin-induced cardioprotection was assessed.

JAK-2

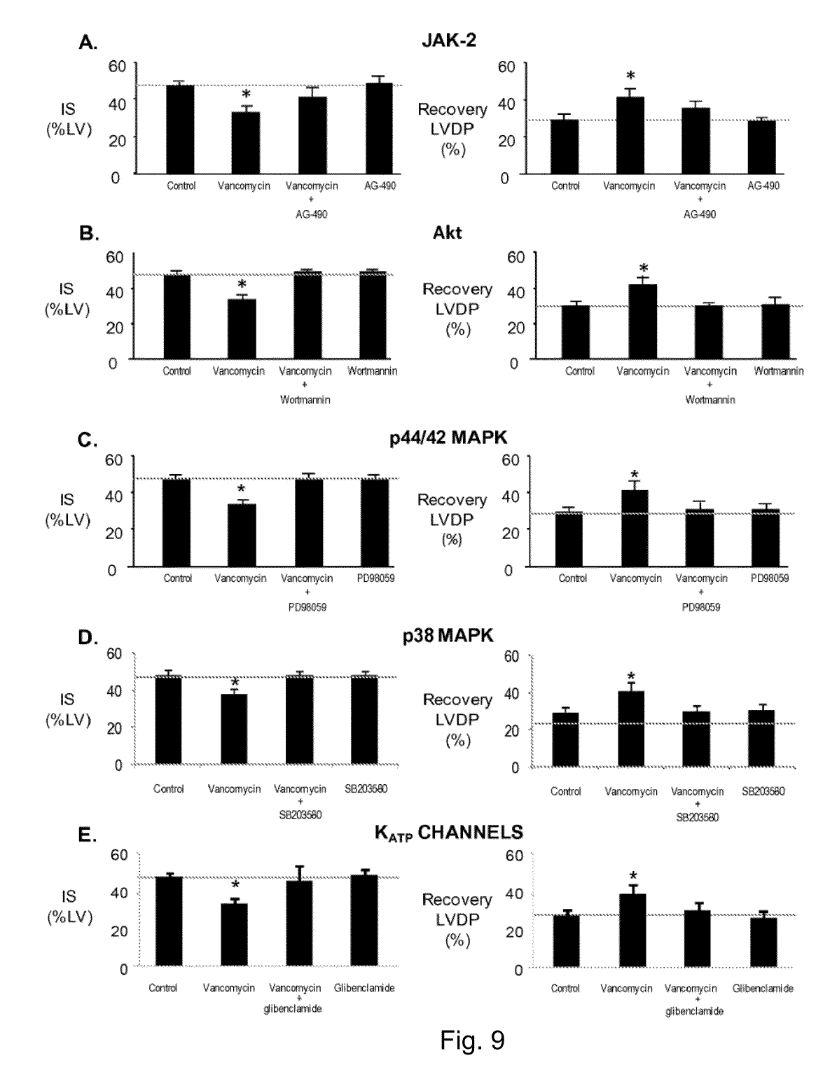

The present disclosure investigates whether microbial metabolites affected by vancomycin bind to a receptor and activate Janus kinase (JAK). Hearts were isolated from vancomycin-treated rats and perfused with a JAK-2 inhibitor AG-490 (1 μM) prior to in vitro ischemia/reperfusion as described in example 3. AG-490 partially abolished the ability of vancomycin to reduce myocardial necrosis and to enhance ventricular function following ischemia/reperfusion. AG-490 alone had no effect on cardioprotection (FIG. 9A). The results presented herein suggest JAK-2 signaling contributes to vancomycin mediated cardioprotection.

Akt

The present disclosure investigates whether vancomycin-induced cardioprotection is mediated by Akt. Isolated hearts were perfused with an Akt/PI3 kinase inhibitor Wortmannin prior to in vitro ischemia/reperfusion as described in example 3. Wortmannin (100 nM) abolished the ability of vancomycin to reduce myocardial necrosis and to enhance ventricular function following ischemia/reperfusion. Wortmannin alone had no effect on cardioprotection (FIG. 9B). The results presented herein suggest Akt signaling contributes to vancomycin mediated cardioprotection.

p42/44 MAPK

The present disclosure investigates whether vancomycin-induced cardioprotection is mediated by p42/44 MAPK. Hearts were perfused with a p42/44 MAPK inhibitor PD98059 prior to in vitro ischemia/reperfusion as described in example 3. PD98059 (10 μM) abrogated the ability of vancomycin to reduce myocardial necrosis and to enhance ventricular function following ischemia/reperfusion. PD98059 alone had no effect on cardioprotection (FIG. 9C). The results presented herein suggest p42/44 MAPK signaling contributes to vancomycin mediated cardioprotection.

p38 MAPK

The present disclosure investigates whether vancomycin-induced cardioprotection is mediated by p38 MAPK. Isolated hearts were perfused with a p38 MAPK inhibitor SB 203580 prior to in vitro ischemia/reperfusion as described in example 3. SB 203580 (15 μM) abolished the ability of vancomycin to reduce myocardial necrosis and to enhance ventricular function following ischemia/reperfusion. SB203580 alone had no effect on cardioprotection (FIG. 9D). The results presented herein suggest p38 MAPK signaling contributes to vancomycin mediated cardioprotection.

K_{ATP} Channels

The present disclosure investigates whether vancomycin-induced cardioprotection is mediated by K_{ATP} channels. Hearts were perfused with a non-selective K_{ATP} channel blocker glibenclamide prior to in vitro ischemia/reperfusion as described in example 3. Glibenclamide (3 μM) abolished the ability of vancomycin to reduce myocardial necrosis and to enhance ventricular function following ischemia/reperfusion. Glibenclamide alone had no effect on cardioprotection (FIG. 9E). The results presented herein suggest signaling via K_{ATP} channels contributes to vancomycin mediated cardioprotection.

### Example 6

### Effect of TPO on Vancomycin Induced Cardioprotection

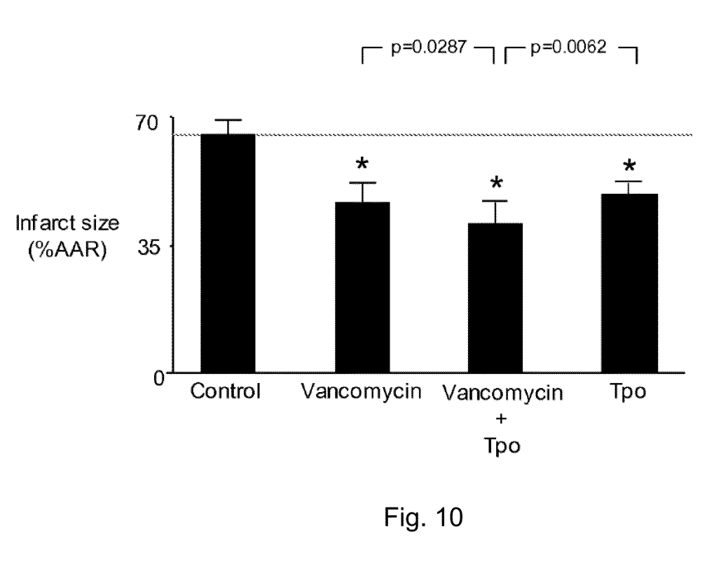

The following example investigates whether vancomycin confers cardioprotection by a mechanism distinct from classical pharmacologic preconditioning with agents such as thrombopoietin (Baker J. E. et al. “Human thrombopoietin reduces myocardial infarct size, apoptosis, and stunning following ischaemia/reperfusion in rats.” Cardiovasc Res 77(1):44-53, 2008). Thrombopoietin confers pharmacological preconditioning through activation of JAK-2, p42/44 MAPK and opening K_{ATP} channels (Baker et al., Cardiovasc Res 77(1):44-53, 2008). Thrombopoietin was administered intravenously in vivo to vancomycin-treated rats prior to ischemia/reperfusion as described in example 3. Vancomycin alone and thrombopoietin alone decreased infarct size by 28% and 25%, respectively. Treatment with vancomycin and thrombopoietin combined decreased infarct size by 38% (FIG. 10). The present disclosure demonstrates that a combination of vancomycin and thrombopoietin conferred a greater reduction in infarct size than with thrombopoietin or vancomycin alone, suggesting that the cardioprotective mechanism of vancomycin may be at least partially distinct from the cardioprotective mechanism of thrombopoietin.

### Example 7

### Effects of Genetic Background on Bacterial Composition

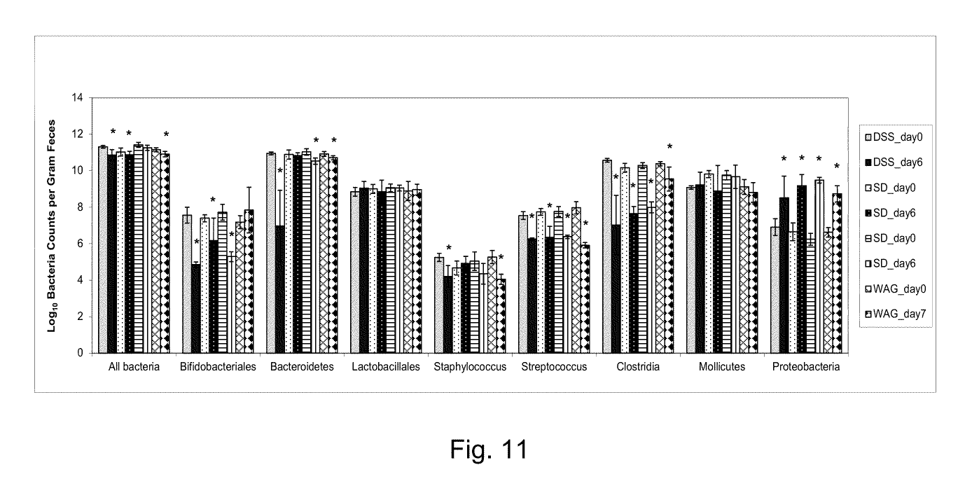

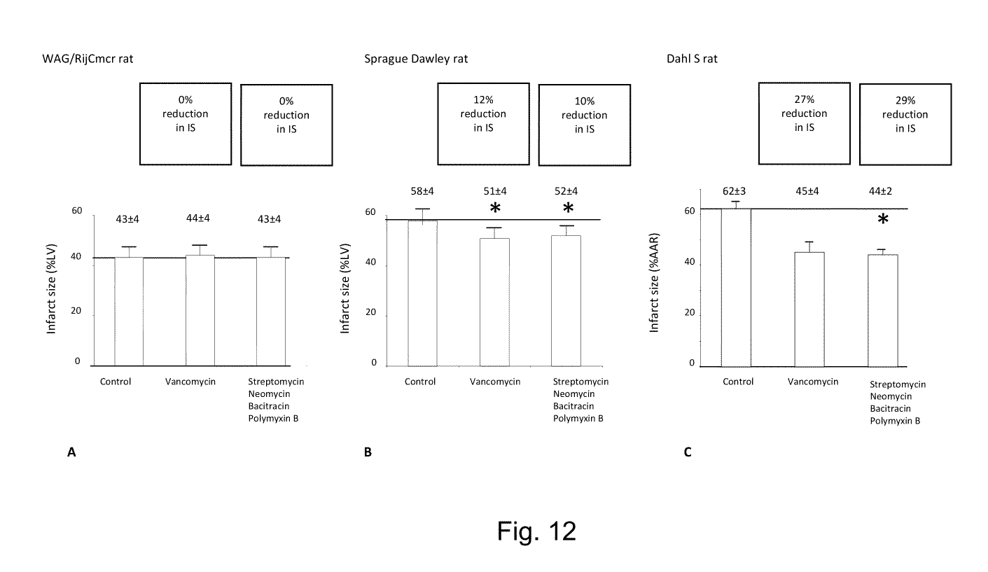

The present example investigates the effect of genetic background on microbiome composition in rats. Composition of intestinal microbiota among healthy people is influenced by host genotype, and examples presented herein suggest that that organic drugs (especially antibiotics) can modulate microbiome composition and host phenotype. Host genetics affect broad intestinal microbiome structure as evidenced by widely differing species compositions of various animal species. Little is understood about the impact of host genome on intestinal microbiota and susceptibility to myocardial infarction. Data presented in FIG. 11 of vancomycin treated rats fed the same diet according to the protocol of example 1 suggests that genetic background contributes to response to antibiotic treatment. Additionally, when environmental influences are minimized, vancomycin treatment or treatment with an antibiotic mixture of streptomycin, neomycin, bacitracin and polymyxin B decreased susceptibility to injury from myocardial ischemia/reperfusion assayed according to the protocol of example 3 in Dahl S rats compared with Sprague Dawley rats, while WAG/RijCmcr rats were unaffected (FIG. 12). Inbred Brown Norway, Fawn Hooded and T2DN rats can additionally be tested. These findings suggest an underlying genomic basis for differing responses to an antibiotic. WAG/RijCmcr rats are resistant and Dahl S rats are sensitive to vancomycin with an intermediate response in Sprague Dawley rats. In support of this notion, it has been shown that inbred rat strains exhibit different susceptibilities to injury from myocardial infarction (Baker, J. et al., 2000).

### Example 8

### Effects of Antibiotics on Microbiota

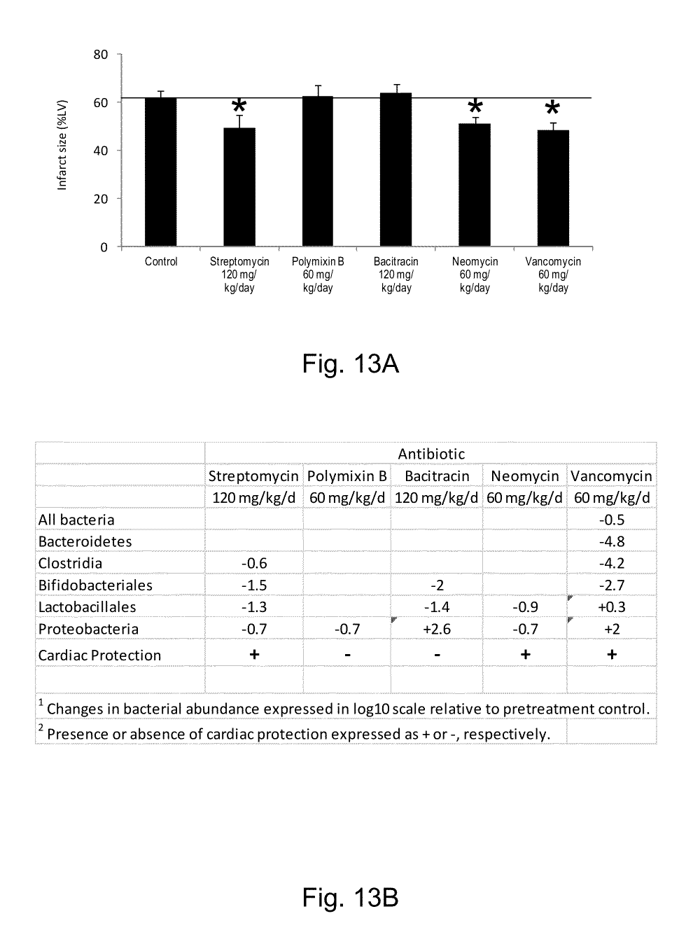

In this example, the effects of antibiotics other than vancomycin on myocardial infarct size are investigated. Two other non-absorbed antibiotics, neomycin and streptomycin, reduce myocardial infarct size as assayed using the techniques of example 3. Other non-absorbed antibiotics, polymyxin B, and bacitracin, did not induce protection in Dahl S rats (FIG. 13A). Antibacterial activity of antibiotics was also characterized using 16S rRNA based PCR as described in example 2. In general, antibiotic treatments were sufficient to reduce abundance of one or more bacterial taxa (FIG. 13B). These results showed antibiotics had differential effects on abundance and diversity of microbiota and these differences contributed to presence or absence of myocardial protection. In rare cases, antibiotic treatment increased abundance of specific taxa, For example, vancomycin can concurrently reduce overall bacterial numbers and increase abundance of Proteobacteria and Lactobacillales because abundance of these taxa only accounted for 0.01 and 1 percent of the bacterial population. No obvious correlation was found between cardiac protection and increase or decrease of any investigated bacterial taxa. Due to the broadly reactive nature of primers used, which detect tens to hundreds of bacterial species, it is suspected that the data conceals changes in abundance of bacterial species that contribute to cardioprotection.

### Example 9

### Method for Monitoring Metabolites

In this example, methods for assaying metabolite levels using mass spectrometry are described. Each sample received was accessioned into the Metabolon Laboratory Information Management System (LIMS) and was assigned by the LIMS a unique identifier, which was associated with the original source identifier only. This identifier was used to track all sample handling, tasks, results etc. The samples (and all derived aliquots) were bar-coded and tracked by the LIMS system. All portions of any sample were automatically assigned their own unique identifiers by the LIMS when a new task was created; the relationship of these samples was also tracked. All samples were maintained at −80° C. until processed.

Sample Preparation

The sample preparation process was carried out using the automated MicroLab STAR® system from Hamilton Company. Recovery standards were added prior to the first step in the extraction process for Quality Control (QC) purposes. Sample preparation was conducted using a proprietary series of organic and aqueous extractions to remove the protein fraction while allowing maximum recovery of small molecules. The resulting extract was divided into two fractions; one for analysis by LC and one for analysis by GC. Samples were placed briefly on a TurboVap® (Zymark) to remove the organic solvent. Each sample was then frozen and dried under vacuum. Samples were then prepared for the appropriate instrument, either LC/MS or GC/MS.

Quality Assurance (QA)/QC: For QA/QC purposes, a number of additional samples are included with each day's analysis. Furthermore, a selection of QC compounds is added to every sample, including those under test. These compounds are carefully chosen so as not to interfere with the measurement of the endogenous compounds. Tables 2 and 3 describe the QC samples and compounds. These QC samples are primarily used to evaluate the process control for each study as well as aiding in the data curation.

TABLE 2
Description of Metabolon QC Samples

| Type | Description | Purpose |
|---|---|---|
| MTRX | Large pool of human plasma | Assure that all aspects of |
| | maintained by Metabolon that has | Metabolon process |
| | been characterized extensively. | are operating within |
| | | specifications. |
| CMTRX | Pool created by taking a small | Assess the effect of a |
| | aliquot from every customer | non-plasma matrix on |
| | sample. | the Metabolon process |
| | | and distinguish |
| | | biological variability |
| | | from process variability. |
| PRCS | Aliquot of ultra-pure water | Process Blank used |
| | | to assess |
| | | the contribution to |
| | | compound signals |
| | | from the process. |
| SOLV | Aliquot of solvents used in | Solvent blank used to |
| | extraction. | segregate contamination |
| | | sources in the extraction. |

TABLE 3
Metabolon QC Standards

| Type | Description | Purpose |
|---|---|---|
| DS | Derivatization Standard | Assess variability of derivatization for |
| | | GC/MS samples. |
| IS | Internal Standard | Assess variability and performance |
| | | of instrument. |
| RS | Recovery Standard | Assess variability and verify |
| | | performance of extraction and |
| | | instrumentation. |

Liquid Chromatography/Mass Spectrometry (LC/MS, LC/MS²)

The LC/MS portion of the platform was based on a Waters ACQUITY UPLC and a Thermo-Finnigan LTQ mass spectrometer, which consisted of an electrospray ionization (ESI) source and linear ion-trap (LIT) mass analyzer. The sample extract was split into two aliquots, dried, then reconstituted in acidic or basic LC-compatible solvents, each of which contained 11 or more injection standards at fixed concentrations. One aliquot was analyzed using acidic positive ion optimized conditions and the other using basic negative ion optimized conditions in two independent injections using separate dedicated columns. Extracts reconstituted in acidic conditions were gradient eluted using water and methanol both containing 0.1% Formic acid, while the basic extracts, which also used water/methanol, contained 6.5 mM Ammonium Bicarbonate. The MS analysis alternated between MS and data-dependent MS² scans using dynamic exclusion.

Gas Chromatography/Mass Spectrometry (GC/MS)

The samples destined for GC/MS analysis were re-dried under vacuum desiccation for a minimum of 24 hours prior to being derivatized under dried nitrogen using bistrimethyl-silyl-trifluoroacetamide (BSTFA). The GC column was 5% phenyl and the temperature ramp is from 40° to 300° C. in a 16 minute period. Samples were analyzed on a Thermo-Finnigan Trace DSQ fast-scanning single-quadrupole mass spectrometer using electron impact ionization. The instrument was tuned and calibrated for mass resolution and mass accuracy on a daily basis. The information output from the raw data files was automatically extracted as discussed below.

Accurate Mass Determination and MS/MS Fragmentation (LC/MS), (LC/MS/MS)

The LC/MS portion of the platform was based on a Waters ACQUITY UPLC and a Thermo-Finnigan LTQ-FT mass spectrometer, which had a linear ion-trap (LIT) front end and a Fourier transform ion cyclotron resonance (FT-ICR) mass spectrometer backend. For ions with counts greater than 2 million, an accurate mass measurement could be performed. Accurate mass measurements could be made on the parent ion as well as fragments. The typical mass error was less than 5 ppm. Ions with less than two million counts require a greater amount of effort to characterize. Fragmentation spectra (MS/MS) were typically generated in data dependent manner, but if necessary, targeted MS/MS could be employed, such as in the case of lower level signals.

Bioinformatics

The informatics system consisted of four major components, the Laboratory Information Management System (LIMS), the data extraction and peak-identification software, data processing tools for QC and compound identification, and a collection of information interpretation and visualization tools for use by data analysts. The hardware and software foundations for these informatics components were the LAN backbone, and a database server running Oracle 10.2.0.1 Enterprise Edition.

LIMS

The purpose of the Metabolon LIMS system was to enable fully auditable laboratory automation through a secure, easy to use, and highly specialized system. The scope of the Metabolon LIMS system encompasses sample accessioning, sample preparation and instrumental analysis and reporting and advanced data analysis. All of the subsequent software systems are grounded in the LIMS data structures. It has been modified to leverage and interface with the in-house information extraction and data visualization systems, as well as third party instrumentation and data analysis software.

Data Extraction and Quality Assurance

The data extraction of the raw mass spec data files yielded information that could be loaded into a relational database and manipulated without resorting to BLOB manipulation. Once in the database the information was examined and appropriate QC limits were imposed. Peaks were identified using Metabolon's proprietary peak integration software, and component parts were stored in a separate and specifically designed complex data structure.

Compound Identification

Compounds were identified by comparison to library entries of purified standards or recurrent unknown entities. Identification of known chemical entities was based on comparison to metabolomic library entries of purified standards. The combination of chromatographic properties and mass spectra gave an indication of a match to the specific compound or an isobaric entity. Additional entities could be identified by virtue of their recurrent nature (both chromatographic and mass spectral). These compounds have the potential to be identified by future acquisition of a matching purified standard or by classical structural analysis.

Curation

A variety of curation procedures were carried out to ensure that a high quality data set was made available for statistical analysis and data interpretation. The QC and curation processes were designed to ensure accurate and consistent identification of true chemical entities, and to remove those representing system artifacts, mis-assignments, and background noise.

Metabolon data analysts use proprietary visualization and interpretation software to confirm the consistency of peak identification among the various samples. Library matches for each compound were checked for each sample and corrected if necessary.

Normalization

For studies spanning multiple days, a data normalization step was performed to correct variation resulting from instrument inter-day tuning differences. Essentially, each compound was corrected in run-day blocks by registering the medians to equal one (1.00) and normalizing each data point proportionately (termed the “block correction”). For studies that did not require more than one day of analysis, no normalization is necessary, other than for purposes of data visualization.

Statistical Calculation

For many studies, two types of statistical analysis are usually performed: (1) significance tests and (2) classification analysis. (1) For pair-wise comparisons Welch's t-tests and/or Wilcoxon's rank sum tests are typically performed. For other statistical designs various ANOVA procedures may be performed (e.g., repeated measures ANOVA). (2) For classification random forest analyses is mainly used. Random forests give an estimate of how well individuals can be classified in a new data set into each group, in contrast to a t-test, which tests whether the unknown means for two populations are different or not. Random forests create a set of classification trees based on continual sampling of the experimental units and compounds. Then each observation is classified based on the majority votes from all the classification trees. Statistical analyses are performed with the program “R” http://cran.r-project.org/.

### Example 10

### Role of Microbial Metabolites on Myocardial Infarction

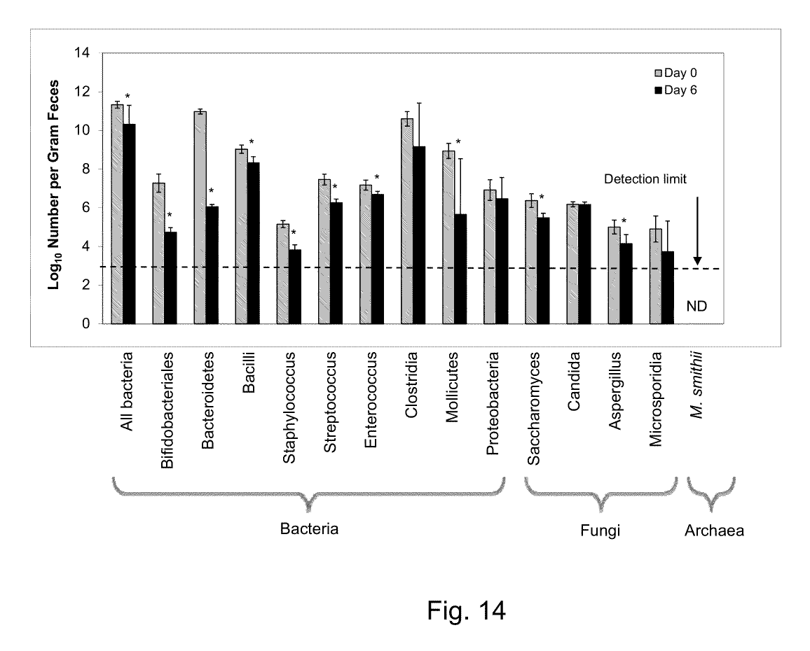

To alter the composition of the intestinal microbiota, a combination of streptomycin (120 mg/kg/day), neomycin (60 mg/kg/day), bacitracin (120 mg/kg/day), and polymyxin B (60 mg/kg/day) were added to the drinking water. The microbial populations present in the feces were monitored by 16S/18S rRNA quantitative RT-PCR using the methods described in examples 1 and 2. The combination of antibiotics reduced total bacterial numbers and altered the abundance of specific microbial species (FIG. 14). The treatments resulted in similar reductions in the various taxa of microbes compared with vancomycin as shown in example 4. Exceptions included the Bacilli group of bacteria which increased two fold in response to vancomycin treatment but decreased five fold in response to the antibiotic mixture and the Proteobacteria which increased 120 fold in response to vancomycin treatment but did not change in response to the antibiotic mixture. Bacterial densities were log₁₀ transformed, and a paired, 2-tailed, t test was used to determine the significance of any differences. Data reported are means±SD. Statistical analysis was performed by use of the paired, 2-tailed, t test. Significance was set at P<0.05.

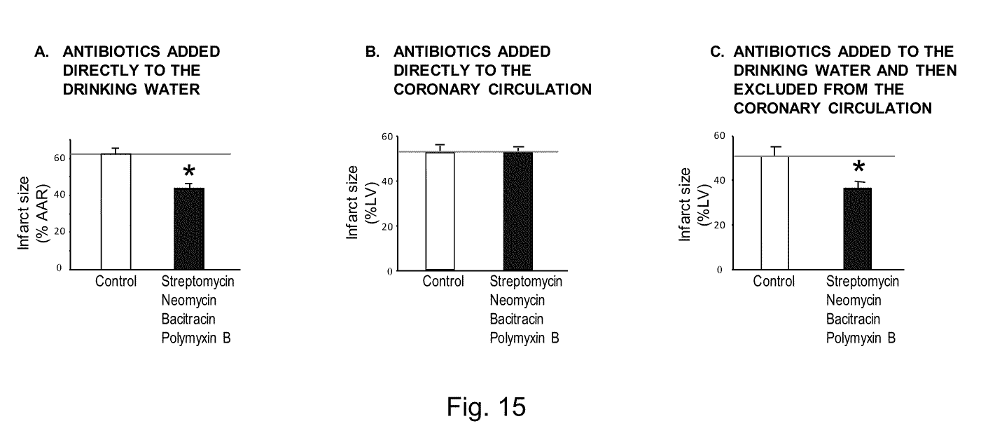

Infarct size was measured in rats treated with the antibiotic mixture using the method described in example 3. The antibiotic mixture decreased infarct size by 29% (FIG. 15A). To determine whether the decrease in infarct size was a direct effect of antibiotics present in the coronary vasculature, blood levels of streptomycin, neomycin, bacitracin and polymyxin B were measured. The level of these antibiotics in the blood was below the detection limits of the assays used (1 μM). When these antibiotics were added directly to the coronary perfusate at a concentration of 1 μM in an in vitro model of myocardial ischemia/reperfusion, as described in example 3, there was no reduction in infarct size (FIG. 15B). To determine further whether the decrease in infarct size was indirect, antibiotics were added to the drinking water and then excluded from the coronary perfusate in an in vitro model of ischemia/reperfusion. The combination of antibiotics decreased infarct size by 29% (FIG. 15C). The present disclosure therefore indicates a reduction in infarct size despite the absence of the antibiotics in the coronary perfusate at the time of ischemia/reperfusion.

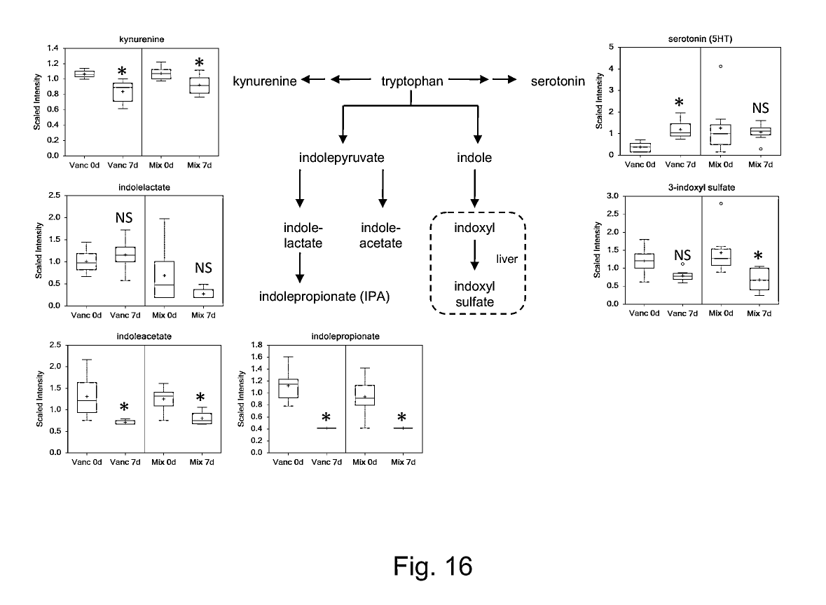

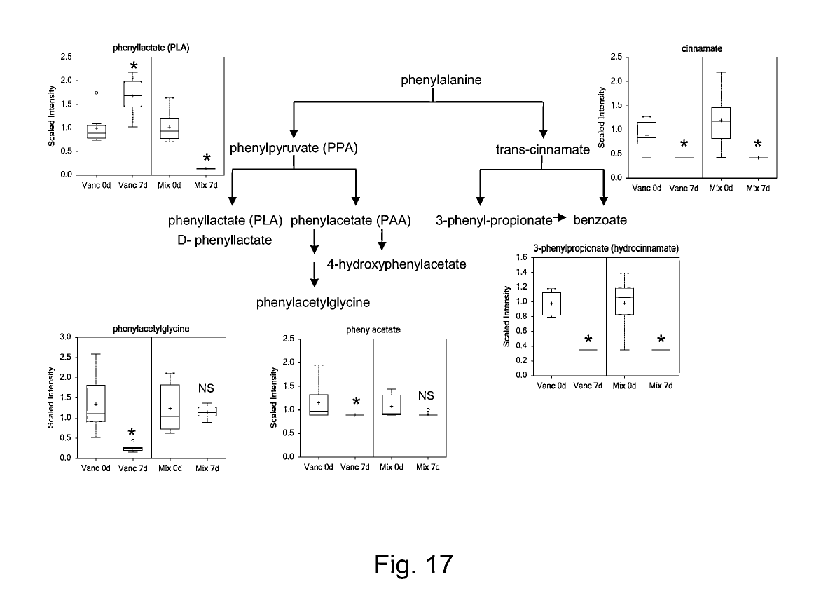

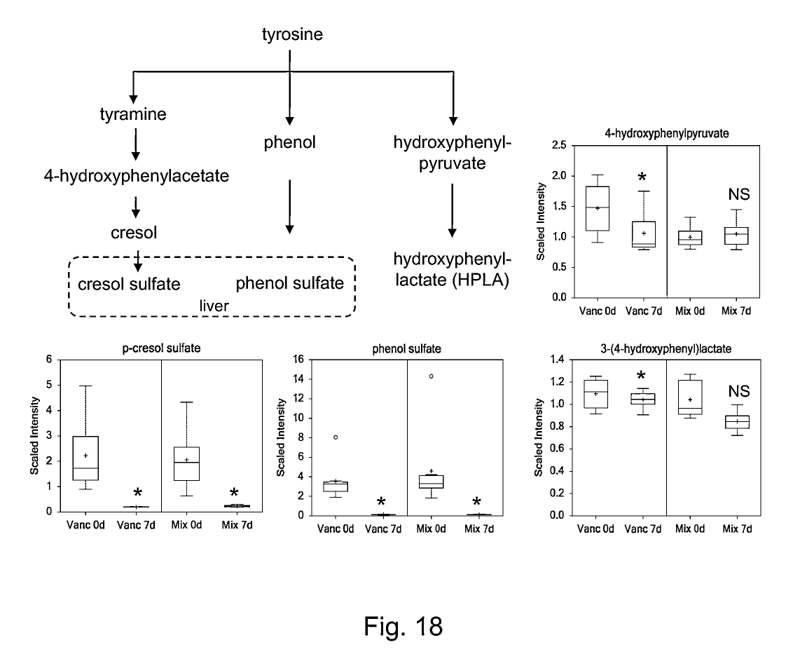

A metabolomic approach was used to identify metabolites in the blood of rats treated for 7 days with vancomycin or a combination of streptomycin (120 mg/kg/day), neomycin (60 mg/kg/day), bacitracin (120 mg/kg/day), and polymyxin B (60 mg/kg/day) as described in example 1. Mass spec analysis showed that treatment with vancomycin alone or a mixture of four antibiotics decreased metabolites known to be modified by intestinal microbiota, including multiple breakdown products of essential aromatic amino acids tryptophan (kynurenine, indoleacetate, indolepropionate, and 3-indoxyl sulfate) (FIG. 16); phenylalanine(phenyllactate, phenylacetylglycine, phenylacetate, 3-phenylpropionate, and cinnamate) (FIG. 17); and tyrosine (p-cresol sulfate, phenol sulfate, 3-(4-hydroxyphenyl)lactate, and 4-hydroxyphenylpyruvate) (FIG. 18). Sulfated products of the tryptophan metabolite 3-indoxyl sulfate (FIG. 16) and tyrosine metabolism p-cresol sulfate and phenol sulfate (FIG. 18), which form in the liver, were decreased following both antibiotic treatments (FIG. 18).

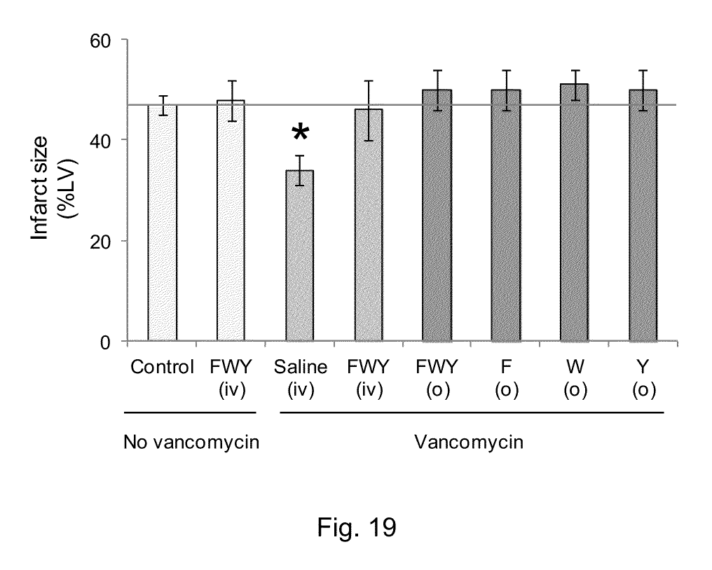

To determine whether decreased levels of circulating amino acid metabolites were associated with a reduction in myocardial infarct size, untreated and vancomycin-treated rats were administered metabolites of phenylalanine (trans-cinnamate [4.50 μg/kg], phenylacetate [4.08 μg/kg], and 3-phenylpropionate [3.06 μg/kg] acids), tryptophan(indole-3-acetate [0.26 μg/kg], 3-indoxyl sulfate [124.50 μg/kg], L-kynurenine [34.99 μg/kg] and 3-indolepropionate [2.73 μg/kg]), and tyrosine (4-hydroxyphenylpyruvate [2.04 μg/kg], and p-hydroxyphenyllactate [3.54 μg/kg]) intravenously at 24 and 12 hours prior to in vitro ischemia/reperfusion studies described in example 3. These doses were selected to reconstitute the metabolite concentrations in the circulation. Metabolites of phenylalanine, tryptophan and tyrosine abolished the decrease in infarct size conferred by vancomycin treatment (FIG. 19). Amino acid metabolite treatment intravenously in the absence of vancomycin pre-treatment had no effect on infarct size (FIG. 19).

To further determine if dietary reconstitution of the metabolites were sufficient to abolish vancomycin-induced reduction in infarct size, the metabolites of all three or each individual aromatic amino acid, phenylalanine, tryptophan and tyrosine, were added to the drinking water of vancomycin-treated rats at the same dosage as above. Oral supplements with metabolites of individual amino acids or in combination abolished the reduction in infarct size (FIG. 19).

The present disclosure demonstrates a proof of concept and a mechanistic link between metabolites of amino acids derived from the intestinal microbiota and the severity of myocardial infarction in the host. Antibiotics were used as tools to temporarily decrease or increase the abundance of specific bacterial groups in the rat intestine, and mass spectrometry was used to profile changes in the abundance of metabolites produced by the microbiota in the blood plasma. Antibiotics reduced the general abundance of the intestinal microbiota and metabolites associated with microbial fermentation of phenylalanine, tyrosine, and tryptophan. Reductions in these metabolites in the circulation were associated with reduced severity of myocardial infarction. This cardioprotective effect is indirect as the antibiotics used are not absorbed into the circulation, and when administered systemically, have no effect on myocardial infarction. Reconstitution of systemic metabolite levels, either through intravenous delivery or oral feeding, abolished the cardioprotective phenotype. These results show that the reach of the metabolites produced by the intestinal microbiota can extend far beyond the local environment of the gut to remote organs such as the heart.

Little is understood of the molecular mechanisms by which metabolites generated by the microbiota influence host physiological functions. The data presented herein suggest a direct effect of the metabolites derived from aromatic amino acids on the myocardium. One possible explanation is that the increased levels of the metabolites in control animals sensitize their myocardium to infarction by increasing oxidative stress on the myocardium's mitochondria. In support of this notion, phenylpropionate and phenylacetate, both phenolic acid metabolites of phenylalanine, increased mitochondrial dysfunction by interfering with NAD-dependent oxidation, increased production of reactive oxygen species, and induced mitochondrial pore opening (Fedotcheva, N. I. et al. Toxic effects of microbial phenolic acids on the functions of mitochondria.” Toxicol. Lett. 180(3):182-188, 2008). However, it is also expected that many metabolic and signaling pathways between different organs including the aromatic amino acid metabolites, leptin signaling, the liver, and the heart can all interact to affect the cardioprotective phenotype.

The present disclosure also demonstrates that vancomycin and Lactobacillus plantarum are not unique in their capability of inducing cardioprotection. The combination of streptomycin, neomycin, bacitracin, and polymixin B also induced an equivalent level of cardioprotection (Lam et al.). Quantitation of the bacterial populations show that the antibiotic combination decreased Bacilli and Proteobacteria abundance as opposed to the increases observed in vancomycin treated rats (Lam et al.). The results presented herein suggests that it is not likely that increased abundance of Bacilli bacteria, such as Lactobacillus plantarum, are responsible for cardioprotection in rats treated with the combination of antibiotics. Similar reductions in aromatic amino acid metabolites observed in both antibiotic combination and vancomycin treatments suggest that bacterial species other than those in the Bacilli and Proteobacteria taxa contribute to cardioprotection. The only metabolite that was different between the treatments was phenyllactate which increased in response to vancomycin treatment. Phenyllactate is an antimicrobial and antifungal that is known to be produced by lactobacillus bacteria, such as Lactobacillus plantarum (Jia, J. et al. “Bioconversion of phenylpyruvate to phenyllactate: gene cloning, expression, and enzymatic characterization of D- and L1-lactate dehydrogenases from Lactobacillus plantarum SK002.” Appl. Biochem. Biotechnol. 162(1):242-251, 2010) and its increase correlates with and support the observed overgrowth of Bacilli bacteria (Lam et al.).

### EQUIVALENTS

Those skilled in the art will recognize, or be able to ascertain using no more than routine experimentation, many equivalents to the specific embodiments of the invention described herein. The scope of the present invention is not intended to be limited to the above Description, but rather is as set forth in the following claims:

What is claimed is:

1. A method of identifying and/or characterizing incidence and/or risk of cardiac defect comprising:
providing a reference microbial signature that correlates with extent or degree of cardiac defect; and
determining a microbial signature present in a microbiota sample from an individual whose incidence and/or risk of cardiac defect is to be identified or characterized.

2. The method of claim 1 further comprising comparing the microbial signature present in a microbiota sample from an individual whose incidence and/or risk of cardiac defect is to be identified or characterized with the reference microbial signature.

3. The method of claim 1 wherein a microbiota sample comprises one or more types of microbes found in a particular organ or tissue of an individual.

4. The method of claim 1 wherein a microbiota sample comprises a set of substantially all types of microbes found in a particular organ or tissue of an individual.

5. The method of claim 3 wherein the particular organ or tissue of an individual is a gastrointestinal tract of an individual.

6. The method of claim 5 wherein the microbiota sample is a fecal sample.

7. The method of claim 1 wherein the individual is human.

8. The method of claim 1 wherein the individual is an animal.

9. The method of claim 1 wherein the microbial signature comprises a level or set of levels of one or more types of microbes or components or products thereof present in a microbiota sample.

10. The method of claim 1 wherein the microbial signature comprises a set of levels of a set of substantially all types of microbes or components or products thereof present in a microbiota sample.

11. The method of claim 9 wherein the microbial signature comprises a level or set of levels of one or more 16S RNA gene sequences present in a microbiota sample.

12. The method of claim 9 wherein the microbial signature comprises a level or set of levels of one or more microbial metabolites present in a microbiota sample.

13. A method of monitoring a patient scheduled to receive or having received a cardiac procedure comprising:
providing a reference microbial signature that correlates with extent or degree of cardiac defect; and
determining a microbial signature present in a microbiota sample from a patient whose incidence and/or risk of cardiac defect is to be identified or characterized.

14. The method of claim 13 further comprising comparing the microbial signature present in a microbiota sample from an individual whose incidence and/or risk of cardiac defect is to be identified or characterized with the reference microbial signature.

15. The method of claim 13 wherein a microbiota sample comprises one or more types of microbes found in a particular organ or tissue of an individual.

16. The method of claim 13 wherein a microbiota sample comprises a set of substantially all types of microbes found in a particular organ or tissue of an individual.

17. The method of claim 15 wherein the particular organ or tissue of an individual is a gastrointestinal tract of an individual.

18. The method of claim 17 wherein the microbiota sample is a fecal sample.

19. The method of claim 13 wherein the individual is human.

20. The method of claims 13 wherein the individual is an animal.

21. The method of claim 13 wherein the microbial signature comprises a level or set of levels of one or more types of microbes or components or products thereof present in a microbiota sample.

22. The method of claim 13 wherein the microbial signature comprises a set of levels of a set of substantially all types of microbes or components or products thereof present in a microbiota sample.

23. The method of claim 21 wherein the microbial signature comprises a level or set of levels of one or more 16S RNA gene sequences present in a microbiota sample.

24. The method of claim 21 wherein the microbial signature comprises a level or set of levels of one or more microbial metabolites present in a microbiota sample.

25. A method of identifying and/or characterizing microbial signatures correlated with incidence and/or risk of cardiac defect comprising:
determining a first set of levels of one or more types of microbes or components or products thereof in a first collection of microbiota samples, where each sample in the first collection of microbiota samples shares a common feature of incidence and/or risk of cardiac defect;
determining a second set of levels of the one or more types of microbes or components or products thereof in a second collection of microbiota samples, which second collection of microbiota samples does not share the common feature of incidence and/or risk of cardiac defect but is otherwise comparable to the first set of microbiota samples;
identifying a microbial signature comprising levels within the first or second set that correlates with presence or absence of the common feature of incidence and/or risk of cardiac defect.

26. The method of claim 25 wherein microbiota samples comprise samples of one or more types of microbes found in particular organs or tissues from which the microbiota samples are collected.

27. The method of claim 25 wherein microbiota samples comprises samples of substantially all types of microbes found in particular organs or tissues from which the microbiota samples are collected.

28. The method of claim 26 wherein the particular organ or tissue is a gastrointestinal tract.

29. The method of claim 28 wherein the microbiota samples are fecal samples.

30. The method of claim 25 wherein a level or set of levels of one or more types of microbes or components or products thereof comprises a level or set of levels of one or more 16S RNA gene sequences present in a microbiota sample.

31. The method of claim 25 wherein a level or set of levels of one or more types of microbes or components or products thereof comprises a level or set of levels of one or more microbial metabolites present in a microbiota sample.

32. The method of claim 25 wherein the microbiota samples are obtained from host organisms and the common feature of incidence and/or risk of cardiac defect comprises incidence of coronary heart disease in host organisms.

33. The method of claim 25 wherein the microbiota samples are obtained from host organisms and the common feature of incidence and/or risk of cardiac defect comprises prior history of myocardial infarction in host organisms.

34. The method of claim 25 wherein the microbiota samples are obtained from host organisms and the common feature of incidence and/or risk of cardiac defect comprises increased susceptibility to ischemia/reperfusion injury in host organisms.

35. The method of claim 25 wherein the common feature of incidence and/or risk of cardiac defect comprises exposure to a microbiome-altering agent having a known correlation with risk of cardiac defect.

36. The method of claim 35 wherein the microbiome-altering agent comprises one or more antibiotics.

37. The method of claim 36, wherein the antibiotics comprise non-absorbable antibiotics.

38. The method of claim 36, wherein the non-absorbable antibiotics comprise vancomycin, neomycin, streptomycin, bacitracin, and/or polymyxin B or combinations thereof.

39. The method of claim 35 wherein the microbiome-altering agent comprises microbes.

40. The method of claim 39, wherein the microbes comprise Lactobacillus plantarum and/or Bifidobacterium lactis.

41. A method of treating or reducing risk for cardiac defect in an individual by altering the microbiome of the individual, the method comprising steps of:
administering to an individual suffering from or susceptible to cardiac defect a microbiome-altering agent, such that the individual's microbiome is altered in a manner that correlates with altered severity of or risk for cardiac defect.

42. The method of claim 41, wherein the microbiome-altering agent comprises one or more antibiotics.

43. The method of claim 42, wherein the antibiotics comprise non-absorbable antibiotics.

44. The method of claim 43, wherein the non-absorbable antibiotics comprise vancomycin, neomycin, streptomycin, bacitracin, and/or polymyxin B or combinations thereof.

45. The method of claim 41, wherein the microbiome-altering agent comprises one or more types of microbes.

46. The method of claim 45, wherein the microbes comprise Lactobacillus plantarum and/or Bifidobacterium lactis.

47. The method of claim 41, wherein the altered severity of or risk for cardiac defect comprises a decrease in leptin levels.

48. The method of claim 41, wherein the altered severity of or risk for cardiac defect comprises a change in levels of one or more microbial metabolites.

49. The method of claim 48, wherein a change in levels of one or more microbial metabolites comprises a decrease in kynurenine, indoleacetate, indolepropionate, 3-indoxyl sulfate, phenyllactate, phenylacetylglycine, phenylacetate, 3-phenylpropionate, cinnamate, p-cresol sulfate, phenol sulfate, 3-(4-hydroxyphenyl)lactate, and/or 4-hydroxyphenylpyruvate or combinations thereof.

50. A composition comprising a microbiome altering agent that, when administered to an individual, alters the individual's microbiome in a manner correlated with reduced severity or risk of cardiac defect.

51. The composition of claim 50, further comprising a pharmaceutically acceptable carrier.

52. The composition of claim 50, provided in or as a food product, functional food or nutraceutical.

53. The composition of claim 50, in a unit dosage form, containing a unit dose amount for administration in accordance with a dosing regimen correlated with achievement of the reduced severity or risk of cardiac defect.

54. The composition of claim 50, wherein the microbiome altering agent is or comprises bacterial cells.

55. The composition of claim 54, provided in or as a food product, functional food or nutraceutical.

56. The composition of claim 54, where the microbiome altering agent is or comprises at least 1,000 bacterial cells.

57. The composition of claim 50 wherein the microbiome altering agent is or comprises an antibiotic.

58. The composition of claim 54, wherein the microbiome altering agent further comprises an antibiotic.
